(19) United States
(12) Patent Application Publication
(10) Pub. No.: US 2024/0003886 A1
(43) Pub. Date: Jan. 4, 2024

(54) DIRECTED PROTEIN EVOLUTION

(71) Applicants: Quantum-Si Incorporated, Branford, CT (US); Marco Ribezzi-Crivellari, Paris (FR); Andrew Griffiths, Paris (FR); Adeline Pichard-Kostuch, Paris (FR); Andrea Flamm, Paris (FR)

(72) Inventors: Marco Ribezzi-Crivellari, Paris (FR); Andrew Griffiths, Paris (FR); Adeline Pichard-Kostuch, Paris (FR); Andrea Flamm, Paris (FR); Lisanne Spenkelink, West Wollongong (AU); Antonius M. van Oijen, Wollongong (AU)

(73) Assignee: Quantum-Si Incorporated, Guilford, CT (US)

(21) Appl. No.: 18/335,924

(22) Filed: Jun. 15, 2023

Related U.S. Application Data
(60) Provisional application No. 63/427,070, filed on Nov. 21, 2022.
(60) Provisional application No. 63/352,621, filed on Jun. 15, 2022.

(51) Int. Cl. G01N 33/573; C12N 15/10; G01N 33/53; G01N 33/58; G01N 33/68

(52) U.S. Cl. CPC G01N 33/573; C12N 15/1065; G01N 2333/91245; G01N 33/582; G01N 33/6854; G01N 33/5308

(57) ABSTRACT

Aspects of the disclosure provide methods and compositions for determining the identity of an analyte using molecular barcodes and single-molecule directed evolution of target biomolecules (e.g., proteins or aptamers).

### CROSS-REFERENCE TO RELATED APPLICATIONS

This application claims priority under 35 U.S.C. § 119(e) to U.S. Provisional Patent Application No. 63/352,621, filed on Jun. 15, 2022; and U.S. Provisional Patent Application No. 63/427,070, filed on Nov. 21, 2022; the content of each of which is hereby incorporated by reference in its entirety.

### REFERENCE TO AN ELECTRONIC SEQUENCE LISTING

The contents of the electronic sequence listing (R070870154US02-SEQ-RJP.xml; Size: 31,103 bytes; and Date of Creation: Jun. 15, 2023) is herein incorporated by reference in its entirety.

### BACKGROUND

Advancements in directed protein evolution technologies have made it possible to conduct high-throughput, bulk experiments to identify nucleic acids and proteins with desired properties. However, these technologies face limitations similar to those encountered by other bulk technologies.

### SUMMARY

In some aspects, the disclosure provides methods and compositions to identify novel proteins or nucleic acids having specific properties, or nucleic acids coding for proteins having specific properties, based on single-molecule measurements. In some aspects, the disclosure provides methods and compositions to identify nucleic acids coding for polymerases having desired properties, based on single-molecule measurements.

Aspects of the present disclosure relate to a method comprising: contacting an analyte with a first barcode recognition molecule and a second barcode recognition molecule, wherein the analyte is connected to a barcode comprising a first nucleic acid index sequence and a second nucleic acid index sequence, wherein the first barcode recognition molecule specifically binds to the first nucleic acid index sequence and the second barcode recognition molecule specifically binds to the second nucleic acid index sequence; detecting a series of signal pulses indicative of binding interactions between (i) the first barcode recognition molecule and the first nucleic acid index sequence, and (ii) the second barcode recognition molecule and the second nucleic acid index sequence; and determining the identity of the analyte based on the series of signal pulses.

Aspects of the present disclosure relate to a method comprising: contacting an analyte with a first barcode recognition molecule and a second barcode recognition molecule, wherein the analyte is connected to a barcode comprising a first nucleic acid index sequence, wherein the first barcode recognition molecule and the second barcode recognition molecule specifically bind to the first nucleic acid index sequence, wherein either (a) the first barcode recognition molecule binds to the first nucleic acid index sequence with a different affinity than the second barcode recognition molecule binds to the first nucleic acid index sequence or (b) the first barcode recognition molecule comprises a first detectable label and the second barcode recognition molecule comprises a second detectable label; detecting a series of signal pulses indicative of binding interactions between (i) the first barcode recognition molecule and the first nucleic acid index sequence, and (ii) the second barcode recognition molecule and the first nucleic acid index sequence; and determining the identity of the analyte based on the series of signal pulses.

Aspects of the present disclosure relate to a method comprising: (i) contacting an analyte with a first barcode recognition molecule and a second barcode recognition molecule, wherein the analyte is connected to a double-stranded barcode comprising a first nucleic acid index sequence and a second nucleic acid index sequence, wherein the first barcode recognition molecule specifically binds to the first nucleic acid index sequence and the second barcode recognition molecule specifically binds to the second nucleic acid index sequence; (ii) detecting a series of signal pulses indicative of binding interactions between (i) the first barcode recognition molecule and the first nucleic acid index sequence, and (ii) the second barcode recognition molecule and the second nucleic acid index sequence; and (iii) determining the identity of the analyte based on the series of signal pulses.

Aspects of the present disclosure relate to a method comprising: (i) contacting an analyte with a first barcode recognition molecule, wherein the analyte is connected to a double-stranded barcode comprising a first nucleic acid index sequence and a second nucleic acid index sequence, wherein the first barcode recognition molecule specifically binds to the first nucleic acid index sequence and specifically binds to the second nucleic acid index sequence; (ii) detecting a series of signal pulses indicative of binding interactions between (i) the first barcode recognition molecule and the first nucleic acid index sequence, and (ii) the first barcode recognition molecule and the second nucleic acid index sequence; and (iii) determining the identity of the analyte based on the series of signal pulses.

In some embodiments, prior to (i), one or more segments of the nucleic acid strand that is bound to the index sequences are removed from the double-stranded barcode, optionally wherein this contacting step is performed in the presence of single-stranded binding (SSB) protein. In some embodiments, the one or more segments of the nucleic acid strand that is bound to the index sequences are removed from the double-stranded barcode using incubation with enzymes or chemical means. In some embodiments, prior to (i), the double-stranded barcode is contacted with a nicking enzyme to remove one or more segments of the nucleic acid strands that is bound to the index sequences, optionally wherein the double-stranded barcode comprises restriction sites surrounding the index sequences that are recognized by the nicking enzyme, optionally wherein this contacting step is performed in the presence of single-stranded binding (SSB) protein.

In some embodiments, the SSB protein is a herpes simplex virus (HSV-1) single-strand DNA-binding protein, a bacterial SSB, replication protein A, or Eukaryotic mitochondrial SSB. In some embodiments, the analyte is a DNA molecule, an RNA molecule, a polypeptide, a protein, or a nucleic acid aptamer. In some embodiments, the first nucleic acid index sequence and the second nucleic acid index sequence comprise different nucleotide sequences and/or different nucleic acid modifications.

In some embodiments, the first nucleic acid index sequence and/or the second nucleic acid index sequence comprises a length of 4-15 nucleotides, 5-10 nucleotides, 6-9 nucleotides, 6 nucleotides, 7 nucleotides, 8 nucleotides, or 9 nucleotides. In some embodiments, the nucleotide sequence of the second nucleic acid index sequence comprises one nucleobase substitution relative to the nucleotide sequence of the first nucleic acid index sequence. In some embodiments, the barcode comprises a spacer between the first nucleic acid index sequence and the second nucleic acid index sequence. In some embodiments, the spacer is a non-nucleic acid spacer or a nucleic acid spacer, optionally wherein the non-nucleic acid spacer is a polyethylene glycol spacer. In some embodiments, the nucleic acid spacer comprises a length of 5-35 nucleotides, 10-25 nucleotides, 15-30 nucleotides, 10-20 nucleotides, 10, 11, 12, 13, 14, 15, 16, 17, 18, 19, 20, or 25 nucleotides. In some embodiments, the barcode further comprises a third nucleic acid index sequence, optionally wherein the third nucleic acid index sequence comprises two nucleobase substitutions relative to the nucleotide sequence of the first nucleic acid index sequence. In some embodiments, the first barcode recognition molecule specifically binds to the third nucleic acid index sequence. In some embodiments, the analyte is contacted with a third barcode recognition molecule, wherein the third barcode recognition molecule specifically binds to the third nucleic acid index sequence. In some embodiments, the barcode further comprises a fourth nucleic acid index sequence, optionally wherein the fourth nucleic acid index sequence comprises three nucleobase substitutions relative to the nucleotide sequence of the first nucleic acid index sequence. In some embodiments, the first barcode recognition molecule specifically binds to the fourth nucleic acid index sequence. In some embodiments, the analyte is contacted with a fourth barcode recognition molecule, wherein the fourth barcode recognition molecule specifically binds to the fourth nucleic acid index sequence.

In some embodiments, the signal pulse comprises a pulse duration that is characteristic of a dissociation rate of binding between the barcode recognition molecule and a nucleic acid index sequence. In some embodiments, at least one signal pulse is separated from another by an interpulse duration that is characteristic of an association rate of barcode recognition molecule binding.

In some embodiments, the barcode recognition molecule is an oligonucleotide probe, a nucleic acid aptamer, or a protein. In some embodiments, the oligonucleotide probe comprises a length of 4-15 nucleotides, 5-10 nucleotides, 6-9 nucleotides, 6 nucleotides, 7 nucleotides, 8 nucleotides, or 9 nucleotides. In some embodiments, at least one of the barcode recognition molecules comprises a detectable label.

In some embodiments, the detectable label is a luminescent label, a fluorescent label, or a conductivity label. In some embodiments, the luminescent label is a fluorophore or a dye. In some embodiments, the first barcode recognition molecule, the second barcode recognition molecule, the third barcode recognition molecule and/or the fourth barcode recognition molecule comprise different detectable labels. In some embodiments, the first barcode recognition molecule and the second barcode recognition molecule comprise different detectable labels. In some embodiments, the detectable labels of the first barcode recognition molecule and the second barcode recognition molecule are fluorophores, and wherein the fluorophore of the first barcode recognition molecule and the fluorophore of the second barcode recognition molecule comprise different absorption/emission spectral properties, optionally wherein the fluorophore of the first barcode recognition molecule and the fluorophore of the second barcode recognition molecule produce different colors.

In some embodiments, at least one of the barcode recognition molecules further comprises a quencher molecule. In some embodiments, the quencher molecule quenches the signal from the detectable label when the barcode recognition molecule is not bound to a nucleic acid index sequence, but does not quench the signal from the detectable label when the barcode recognition molecule is bound to a nucleic acid index sequence.

In some embodiments, the series of signal pulses is a series of real-time signal pulses. In some embodiments, the analyte is attached to a surface, optionally a glass or silica-based surface. In some embodiments, the surface is a surface of a well of a multi-well plate, optionally a 96-well plate or a 384-well plate. In some embodiments, the analyte is covalently or non-covalently attached to the surface. In some embodiments, the analyte is attached to the surface via a secondary molecule or species, optionally via a streptavidin-biotin linkage or by hybridization to a capture oligonucleotide probe covalently linked to the surface.

Aspects of the present disclosure relate to a method comprising: (i) attaching a biomolecule to a surface, wherein the biomolecule comprises (a) a protein and a barcode comprising a first nucleic acid index sequence, or (b) an aptamer and a barcode comprising a first nucleic acid index sequence; (ii) performing a phenotypic assay to determine one or more characteristics of the protein or aptamer; (iii) contacting the biomolecule with a first barcode recognition molecule, wherein the first barcode recognition molecule specifically binds to the first nucleic acid index sequence; (iv) detecting a series of signal pulses indicative of binding interactions between the first barcode recognition molecule and the first nucleic acid index sequence; (v) determining the identity of the biomolecule based on the series of signal pulses.

Aspects of the present disclosure relate to a method comprising: (i) attaching a biomolecule to a surface, wherein the biomolecule comprises (a) a nucleic acid comprising a coding sequence encoding a protein and a molecular barcode comprising a first nucleic acid index sequence, and (b) an amino acid sequence of the protein; (ii) performing a phenotypic assay to determine one or more characteristics of the protein; (iii) contacting the biomolecule with a first barcode recognition molecule, wherein the first barcode recognition molecule specifically binds to the first nucleic acid index sequence; (iv) detecting a series of signal pulses indicative of binding interactions between the first barcode recognition molecule and the first nucleic acid index sequence; (v) determining the identity of the biomolecule based on the series of signal pulses.

In some embodiments, the barcode further comprises a second nucleic acid index sequence. In some embodiments, the first barcode recognition molecule specifically binds to the second nucleic acid index sequence. In some embodiments, the biomolecule is contacted with a second barcode recognition molecule in (iii), wherein the second barcode recognition molecule specifically binds to the second nucleic acid index sequence. In some embodiments, the first nucleic acid index sequence and the second nucleic acid index sequence comprise different nucleotide sequences and/or different nucleic acid modifications. In some embodiments, the first nucleic acid index sequence and/or the second nucleic acid index sequence comprises a length of 4-15 nucleotides, 5-10 nucleotides, 6-9 nucleotides, 6 nucleotides, 7 nucleotides, 8 nucleotides, or 9 nucleotides. In some embodiments, the nucleotide sequence of the second nucleic acid index sequence comprises one nucleobase substitution relative to the nucleotide sequence of the first nucleic acid index sequence. In some embodiments, the barcode comprises a spacer between the first nucleic acid index sequence and the second nucleic acid index sequence. In some embodiments, the spacer is a non-nucleic acid spacer or a nucleic acid spacer, optionally wherein the non-nucleic acid spacer is a polyethylene glycol spacer. In some embodiments, the nucleic acid spacer comprises a length of 5-35 nucleotides, 10-25 nucleotides, 15-30 nucleotides, 10-20 nucleotides, 10, 11, 12, 13, 14, 15, 16, 17, 18, 19, 20, or 25 nucleotides. In some embodiments, the barcode further comprises a third nucleic acid index sequence, optionally wherein the third nucleic acid index sequence comprises two nucleobase substitutions relative to the nucleotide sequence of the first nucleic acid index sequence. In some embodiments, the first barcode recognition molecule specifically binds to the third nucleic acid index sequence. In some embodiments, the biomolecule is contacted with a third barcode recognition molecule in (iii), wherein the third barcode recognition molecule specifically binds to the third nucleic acid index sequence. In some embodiments, the barcode further comprises a fourth nucleic acid index sequence, optionally wherein the fourth nucleic acid index sequence comprises three nucleobase substitutions relative to the nucleotide sequence of the first nucleic acid index sequence. In some embodiments, the first barcode recognition molecule specifically binds to the fourth nucleic acid index sequence. In some embodiments, the biomolecule is contacted with a fourth barcode recognition molecule in (iii), wherein the fourth barcode recognition molecule specifically binds to the fourth nucleic acid index sequence.

In some embodiments, the signal pulse comprises a pulse duration that is characteristic of a dissociation rate of binding between the barcode recognition molecule and a nucleic acid index sequence. In some embodiments, at least one signal pulse is separated from another by an interpulse duration that is characteristic of an association rate of barcode recognition molecule binding. In some embodiments, the barcode recognition molecule is an oligonucleotide probe, a nucleic acid aptamer, or a protein. In some embodiments, the oligonucleotide probe comprises a length of 4-15 nucleotides, 5-10 nucleotides, 6-9 nucleotides, 6 nucleotides, 7 nucleotides, 8 nucleotides, or 9 nucleotides. In some embodiments, at least one of the barcode recognition molecules comprises a detectable label. In some embodiments, the detectable label is a luminescent label, a fluorescent label, or a conductivity label. In some embodiments, the luminescent label is a fluorophore or a dye.

In some embodiments, the first barcode recognition molecule, the second barcode recognition molecule, the third barcode recognition molecule and/or the fourth barcode recognition molecule comprise different detectable labels. In some embodiments, the first barcode recognition molecule and the second barcode recognition molecule comprise different detectable labels. In some embodiments, the detectable labels of the first barcode recognition molecule and the second barcode recognition molecule are fluorophores, and wherein the fluorophore of the first barcode recognition molecule and the fluorophore of the second barcode recognition molecule comprise different absorption/emission spectral properties, optionally wherein the fluorophore of the first barcode recognition molecule and the fluorophore of the second barcode recognition molecule produce different colors.

In some embodiments, at least one of the barcode recognition molecules further comprises a quencher molecule. In some embodiments, the quencher molecule quenches the signal from the detectable label when the barcode recognition molecule is not bound to a nucleic acid index sequence, but does not quench the signal from the detectable label when the barcode recognition molecule is bound to a nucleic acid index sequence.

In some embodiments, the series of signal pulses is a series of real-time signal pulses. In some embodiments, the surface is a glass or silica-based surface. In some embodiments, the surface is a surface of a well of a multi-well plate, optionally a 96-well plate or a 384-well plate. In some embodiments, the biomolecule is covalently or non-covalently attached to the surface. In some embodiments, the biomolecule is attached to the surface via a secondary molecule or species, optionally via a streptavidin-biotin linkage or by hybridization to a capture oligonucleotide probe covalently linked to the surface. In some embodiments, the biomolecule further comprises a spacer between the protein and the barcode. In some embodiments, the biomolecule further comprises a spacer between the aptamer and the barcode. In some embodiments, the biomolecule further comprises a ribosome attached to the coding sequence (e.g., ribosome display). In some embodiments, the biomolecule allows for display of the protein portion and the nucleic acid portion using Snap display or RNA-DNA-puromycin methodologies. In some embodiments, the coding sequence is attached to the amino acid sequence.

In some embodiments, the phenotypic assay is a binding assay or an enzymatic assay. In some embodiments, the binding assay comprises incubating the biomolecule with an antigen or ligand, optionally a fluorescently labeled antigen or ligand, and determining the binding affinity of the biomolecule for the antigen or ligand. In some embodiments, the enzymatic assay comprises incubating the biomolecule with substrate, and determining the ability of the biomolecule to chemically convert said substrate and/or determining the enzymatic activity of the biomolecule. In some embodiments, the one or more characteristics of the protein or aptamer comprise binding affinity for an antigen, binding kinetics, stability of the protein or aptamer, thermal stability of the protein or aptamer, and/or enzymatic activity number.

Aspects of the present disclosure relate to a method of screening protein variants and/or aptamer variants comprising: (i) generating a library of biomolecules, wherein each of the biomolecules comprises (a) a protein variant and a molecular barcode comprising a first nucleic acid index sequence, or (b) an aptamer variant and a molecular barcode comprising a first nucleic acid index sequence, wherein each of the biomolecules comprises a unique combination of first nucleic acid index sequences; (ii) attaching each of the biomolecules to a surface; (iii) performing a phenotypic assay to determine one or more characteristics of each of the protein variants; (iv) contacting each of the biomolecules with a first barcode recognition molecule, wherein the first barcode recognition molecule specifically binds to the first nucleic acid index sequence; (v) detecting a series of signal pulses indicative of binding interactions between the first barcode recognition molecule and the first nucleic acid index sequence; (vi) determining the identity of each of the biomolecules based on the series of signal pulses; and (vii) identifying biomolecules comprising unique protein variants having one or more desired characteristics. In some embodiments, the library comprises 5-1000, 5-500, 5-100, 10-100, 100-1000, or 50-500 unique biomolecules.

In some embodiments, the analyte or biomolecule is contacted with the first barcode recognition molecule and the second barcode recognition molecule simultaneously.

In some embodiments, the analyte or biomolecule is contacted with all of the barcode recognition molecules simultaneously.

In some embodiments, the analyte or biomolecule is (a) first contacted with the first barcode recognition molecule and the series of signal pulses indicative of binding interactions between the first barcode recognition molecule and the nucleic acid index sequence(s) are detected; and (b) subsequently contacted with the second barcode recognition molecule and the series of signal pulses indicative of binding interactions between the second barcode recognition molecule and the nucleic acid index sequence(s) are detected.

Aspects of the present disclosure relate to a nucleic acid barcode comprising two, three, or four nucleic acid index sequences, wherein each of the nucleic acid index sequences is independently selected from any one of SEQ ID NOs: 25-28 or 36-59.

Aspects of the present disclosure relate to a nucleic acid barcode comprising a first nucleic acid index sequence, a second nucleic acid index sequence, a third nucleic acid index sequence, and a fourth nucleic acid index sequence, wherein each of the nucleic acid index sequences comprises at least 6 nucleotides in length, and wherein the second nucleic acid index sequence comprises one nucleobase substitution relative to the first nucleic acid index sequence, the third nucleic acid index sequence comprises two nucleobase substitutions relative to the first nucleic acid index sequence, and the fourth nucleic acid index sequence comprises three nucleobase substitutions relative to the first nucleic acid index sequence.

Aspects of the present disclosure relate to a nucleic acid barcode comprising a first nucleic acid index sequence, a second nucleic acid index sequence, a third nucleic acid index sequence, and a fourth nucleic acid index sequence, wherein each of the nucleic acid index sequences is complementary to a barcode recognition molecule, wherein the residence time for a binding interaction between the second nucleic acid index sequence and the barcode recognition molecule is at least 2-fold greater than the binding interaction between the first nucleic acid index sequence and the barcode recognition molecule, wherein the residence time for a binding interaction between the third nucleic acid index sequence and the barcode recognition molecule is at least 2-fold greater than the binding interaction between the second nucleic acid index sequence and the barcode recognition molecule, and wherein the residence time for a binding interaction between the fourth nucleic acid index sequence and the barcode recognition molecule is at least 2-fold greater than the binding interaction between the fourth nucleic acid index sequence and the barcode recognition molecule. In some embodiments, the residence time for a binding interaction between the second nucleic acid index sequence and the barcode recognition molecule is 2-fold to 5-fold greater than the binding interaction between the first nucleic acid index sequence and the barcode recognition molecule, wherein the residence time for a binding interaction between the third nucleic acid index sequence and the barcode recognition molecule is 2-fold to 5-fold greater than the binding interaction between the second nucleic acid index sequence and the barcode recognition molecule, and wherein the residence time for a binding interaction between the fourth nucleic acid index sequence and the barcode recognition molecule is 2-fold to 5-fold greater than the binding interaction between the fourth nucleic acid index sequence and the barcode recognition molecule. In some embodiments, the residence time for a binding interaction between the second nucleic acid index sequence and the barcode recognition molecule is 2-fold, 3-fold, 4-fold, or 5-fold greater than the binding interaction between the first nucleic acid index sequence and the barcode recognition molecule, wherein the residence time for a binding interaction between the third nucleic acid index sequence and the barcode recognition molecule is 2-fold, 3-fold, 4-fold, or 5-fold greater than the binding interaction between the second nucleic acid index sequence and the barcode recognition molecule, and wherein the residence time for a binding interaction between the fourth nucleic acid index sequence and the barcode recognition molecule is 2-fold, 3-fold, 4-fold, or 5-fold greater than the binding interaction between the fourth nucleic acid index sequence and the barcode recognition molecule.

In some embodiments, nucleic acid index sequences comprise a length of 4-15 nucleotides, 5-10 nucleotides, 6-9 nucleotides, 6 nucleotides, 7 nucleotides, 8 nucleotides, or 9 nucleotides. In some embodiments, the barcode comprises a spacer between the first nucleic acid index sequence and the second nucleic acid index sequence. In some embodiments, the spacer is a non-nucleic acid spacer, optionally a polyethylene glycol spacer, or a nucleic acid spacer. In some embodiments, the nucleic acid spacer comprises a length of 5-25 nucleotides, 10-20 nucleotides, 10, 11, 12, 13, 14, 15, 16, 17, 18, 19, or 20 nucleotides.

The details of certain embodiments of the invention are set forth in the Detailed Description of Certain Embodiments, as described below. Other features, objects, and advantages of the invention will be apparent from the Examples, Figures, and Claims.

### BRIEF DESCRIPTION OF THE DRAWINGS

The accompanying drawings, which constitute a part of this specification, illustrate several embodiments of the invention and together with the description, serve to explain the principles of the invention.

FIG. 1 shows an examples of signal pulse detection and analysis.

FIG. 2 shows an example of a molecular barcode construct for use in accordance with embodiments of the disclosure.

FIGS. 3A-3B show an example of barcode recognition used in connection with single-cell polypeptide sequencing. FIG. 3A shows a general process in which polypeptides from single cells are labeled with cell-specific barcodes. FIG. 3B generically depicts barcoded polypeptides, which can be analyzed by dynamic sequencing and barcode recognition on a single array substrate.

FIG. 4 shows an example of molecular barcode design. A fluorescently-labeled probe has complementarity to four different nucleic acid index lengths (6 nucleotides to 9 nucleotides). The binding kinetics of the probe to each nucleic acid index depend on the length of the sequence overlap and allow for discrimination and identity of the four nucleic acid index sequences relative to one another.

FIGS. 5A-5B show an example of molecular barcode index binding characteristics. FIG. shows single-stranded nucleic acid barcodes comprising a first nucleic acid index sequence (9 nucleotides in length) tethered to a surface (e.g., a glass coverslip). An exemplary barcode recognition molecule (a fluorescently-labeled complementary oligonucleotide probe) transiently binds to the index sequences. FIG. 5B shows the intensity over time for each barcode interaction with a barcode recognition molecule, generating a series of signal pulses.

FIG. 6 shows an example of molecular barcode assembly performed in the presence of a single-stranded binding (SSB) protein (left) versus an example of molecular barcode assembly performed in the absence of a SSB protein (right). A double-stranded nucleic acid barcode (dsDNA barcode) comprises a first nucleic acid index sequence (10 nucleotides in length) and a second nucleic acid index sequence (10 nucleotides in length) separated by a nucleic acid spacer. The barcode is exposed to a nicking enzyme that generates nicks in one strand of the barcode at restriction sites surrounding the index sequences (e.g., on each side of the index sequences). In the presence of SSB protein, the segments of the nucleic acid bound to the index sequences (“lid” sequences) and nicked by the nicking enzyme are removed/dehybridized to generate a rigid nucleic acid barcode comprising exposed index sequences. In the absence of SSB protein, the single-stranded nucleic acid collapses following nicking and dehybridization.

FIG. 7 shows an example of identification of molecular barcodes described in embodiments of this disclosure. A first barcode recognition molecule (fast kinetics, blue) is capable of binding to a first nucleic acid index sequence of a double-stranded barcode; a second barcode recognition molecule (slow kinetics, red) is capable of binding to a second nucleic acid index sequence of a double-stranded barcode; and a third barcode recognition molecule (slow kinetics, blue) is capable of binding to a third nucleic acid index sequence of a double-stranded barcode to identify whether any given analyte contains said nucleic acid index sequences (thereby enabling identification of the analyte).

FIG. 8 shows a sample workflow of a method for screening for general enzymatic activity, as described in several embodiments of this disclosure. Analytes are encapsulated in an emulsion droplet comprising in vitro transcription translation (IVTT) reagents (1). Following interaction between the analytes and the IVTT reagents, analytes produce proteins or peptides that are encoded by the analyte (2). The protein or peptides are adapted to bind to the analyte via an anchor on the analyte forming a complex (3). The complexes are isolated by size exclusion chromatography (4) and bound to a polymerase (5). The identity of the protein or peptide is determined by a zero-mode waveguide (6) and the sequence of the analyte encoding the protein or peptide is read by the polymerase directly to produce a genotype/phenotype link between the analyte and protein or peptide.

FIG. 9 shows a sample workflow of a method for selecting antibodies, as described in several embodiments of this disclosure. Variant genes encoding antibodies (e.g., scFV antibodies) are ligated to molecular barcodes comprising four nucleic acid index sequences, transcribed and translated using ribosome display, and immobilized on a glass slide via hybridization capture. Fluorescently labeled antigens are used to characterize the binding affinities for each variant antibody, and probes specific to the molecular barcodes are used to sequence the variant genes, thus forming a genotype/phenotype link between the variant genes and the antibodies they encode.

FIG. 10 shows a sample workflow of a method for selecting nucleic acid aptamers, as described in several embodiments of this disclosure. The workflow of FIG. 10 is similar to the workflow of FIG. 9 and is directed to variant nucleic acid aptamers.

FIG. 11 shows a sample workflow of a method for selecting polymerases, as described in several embodiments of this disclosure. Variant genes encoding polymerases are encapsulated in an emulsion droplet comprising IVTT reagents (1). Following interaction between the variant genes and the IVTT reagents, the variant genes produce variant polymerases that are encoded by the variant genes (2). The polymerases are adapted to bind to the nucleic acid variant genes via an anchor on the nucleic acids forming a pool of complexes (3). The emulsion droplets are broken in the presence of heparin, which sequesters variant genes and polymerases that did not form a complex. The complexes are isolated by size exclusion chromatography (4) and analyzed by a zero-mode waveguide (5).

FIG. 12 shows a sample workflow of a method for selecting polymerases using magnetic tweezers, as described in several embodiments of this disclosure. Variant genes are ligated to molecular barcodes and encapsulated in emulsion droplets comprising IVTT reagents (1). Each variant gene contains a hairpin structure with one branch ending in a biotin and the other in a nucleic acid loop (2). The variant genes and the barcodes are separated by a roadblock for polymerization (e.g., a C12 connection). Following encapsulation, the variant genes produce biotinylated polymerases (2) which bind strongly to the variant genes, forming complexes (3). The complexes are bound on magnetic beads (4), remaining material that did not form a complex is removed by washing, and the complexes are transferred to a magnetic tweezers microscope (5). On the magnetic tweezers microscope, the genotypic and phenotypic characteristics are determined for each complex (6).

FIG. 13 shows an example application of the methods directed to directed evolution of polymerases, as described in several embodiments of this disclosure. The workflow in FIG. 13 is similar to the workflow of FIG. 11 with the addition of replication (by Mg²⁺) and digestion of the complexes. Use of an emulsion, in combination with replication and digestion, led to 12-fold enrichment of active polymerase (Q1) over inactive polymerase (Qdead).

FIG. 14 shows an example application of the methods directed to directed evolution of polymerases, as described in several embodiments of this disclosure. The workflow of FIG. 14 is similar to the workflow in FIG. 11 and utilizes active and inactive polymerases.

FIG. 15 shows an example of molecular barcode index binding for identification of a protein analyte (e.g., Cas9). A protein analyte is covalently attached to a nucleic acid barcode comprising four unique indices (Index 1, Index 2, Index 3, and Index 4) on a surface (e.g., a glass coverslip). Fluorescently labeled nucleic acid probes are designed to only bind to one of the unique indices. Binding interactions between probes of different lengths and the unique indices can be used to identify the phenotype of the protein analyte using a workflow as depicted (e.g., Cycle 1 involving injection of Index 1 probes and measurement of fluorescence, Cycle 2 involving injection of Index 2 probes and measurement of fluorescence, etc.).

FIG. 16 shows data depicting the ability of a fluorescently labeled probe (9 nucleotides in length) to bind to a molecular barcode. The frequency and length of ‘on time’ binding events (e.g., wherein ‘on time’ binding refers to the interaction between the probe and the barcode) are provided.

FIG. 17A-17B demonstrate that binding kinetics are dependent on the length of the index sequences. Kinetic signatures of binding provide characteristic distributions of ‘on time’ binding interactions between molecular barcodes and probes of varying length (7, 8, and 9 nucleotides). Provided at right in FIG. 17A is a table of residence times (e.g., ‘on time’ binding) (in seconds) for probes of varying length (7, 8, and 9 nucleotides) binding to discrete barcodes (Barcode sequences A, B, C, D, E, and F).

FIG. 18 shows that the molecular barcoding strategies of the disclosure can be used to correctly identify an analyte comprising a first index sequence and a second index sequence. Two fluorescently labeled probes were used to correctly identify an analyte in 93% of experiments.

FIG. 19 shows that the molecular barcoding strategies of the disclosure can be used to correctly identify target analytes comprising a first index sequence and a second index sequence. The use of two probes was able to correctly identify six protein variants within a sample.

FIGS. 20A-20B show sample workflows of a method (“Gene2Glass”) for linking a genotype with a phenotype, as described in several embodiments of this disclosure. Variant genes are ligated to molecular barcodes comprising nucleic acid index sequences; and may be transcribed and translated using IVTT, biotinylated, and subjected to SNAP display. A nicking process may be used to remove lid sequences prior to incubation with labeled probes to identify the variant gene.

FIG. 21 provides an example method to identify dwell-times (also referred to herein as residence times). A two-state Hidden Markov model may be applied to collected fluorescence data. The resulting dwell-time distribution is then tested against all possible kinetic signatures. One-sided t-tests allow for identification of dwell-times with high confidence.

FIG. 22 demonstrates that ATP is not required during DNA replication.

### DETAILED DESCRIPTION

The present disclosure relates, in part, to novel nucleic acid barcodes comprising one or more nucleic acid index sequences (e.g., a first and a second nucleic acid index sequence) and the use of such nucleic acid barcodes to enable single-molecule identification of analytes such as proteins and aptamers. For example, some aspects of the disclosure provide methods of contacting an analyte (e.g., a protein or an aptamer) that is connected to a barcode comprising one or more nucleic acid index sequences (e.g., a first and a second nucleic acid index sequence) with one or more barcode recognition molecules (e.g., one or more oligonucleotide probes that are complementary to the index sequence(s)) and detecting transient binding interactions between the index sequence(s) and the barcode recognition molecule(s). This detection can, in some embodiments, allow for the identification of the analyte (e.g., on a single-molecule level). In some embodiments, the barcodes are double-stranded and require further processing prior to interactions between the nucleic acid index sequence(s) and the barcode recognition molecule(s), such as incubation of the barcodes with a nicking enzyme to expose the nucleic acid index sequence(s). Further aspects of the disclosure relate to methods of screening protein or aptamer molecules that are associated with (e.g., connected to) a barcode comprising one or more nucleic acid index sequences by combining a phenotypic assay (e.g., a binding assay or an enzymatic assay) with a genotypic assay (e.g., identification of the nucleic acid index sequence(s) by contacting with one or more barcode recognition molecules, and detection of resultant binding interactions).

In some embodiments, the methods described herein that enable single-molecule identification of analytes or biomolecules provide the benefits of (i) diminished costs due to the small amount of reagents employed, (ii) absolute quantification due to the counting of individual molecules and (iii) increased resolution. That increased resolution can for example allow for the determination of the active fraction or percentage of molecules within a sample, and allow for discrimination of a single analyte within a complex mixture.

The present disclosure relates, in part, to methods of identifying nucleic acids having specific properties (phenotypes of interest), or nucleic acids coding for proteins having specific properties, based on single-molecule measurements. In some embodiments, libraries of RNAs or DNAs, which may or may not contain synthetic nucleic acid analogs or nucleic acid modifications, are created. These DNAs and RNAs can code for polypeptides or proteins composed of natural and/or unnatural and/or unusual amino acids. Natural amino acids are amino acids found in natural polypeptide chains. Unnatural amino acids are amino acids not found in natural polypeptide chains. Unusual amino acids are amino acids that are not usually found in natural polypeptide chains, such as citrulline (Cit), hydroxyproline (Hyp), beta-alanine, ornithine (Orn), norleucine (Nle), 3-nitrotyrosine, pyroglutamic acid (Pyr), and nitroarginine. The sequence of each variant of a DNA or RNA may or may not be encoded by a specific sequence barcode. In some embodiments, the barcode can be designed to be optically decoded (e.g., decoded by use of fluorescent probes). In some embodiments, a random or designed library can be created by random mutagenesis or synthetic gene synthesis. In some embodiments, barcodes comprise combinatorial consecutive nucleic acid index sequences (e.g., produced using split-and-pool ligation), e.g., chosen from a limited alphabet of highly dissimilar sequences. In some embodiments, RNA molecules are directly provided or in vitro transcribed from a library of DNA molecules. In some embodiments, the resulting RNA molecules are immobilized on a surface-treated glass slide. In some embodiments the molecules are immobilized by a streptavidin/biotin linkage or by hybridization to a capture probe covalently linked to the slide.

In some embodiments, to screen for protein or aptamer variants, any method for displaying peptides, polypeptides, or proteins on a nucleic acid that codes for them, e.g., any method leading to a stable complex between a nucleic acid, the peptide, polypeptide or protein it codes for and possibly other binding partners, may be performed. For example, in some embodiments, ribosome display can be utilized to generate stable ternary complexes of barcoded mRNA, ribosome and the protein variant encoded by the mRNA. It is also possible to use mRNA display to form stable binary complexes between barcoded mRNAs and the protein variants encoded by the mRNA. Similarly, it is also possible to form a complex between DNA, RNA polymerase, ribosome and protein (see, for example, US 2016/0097050, which is incorporated by reference in its entirety). These complexes can be coupled to the slide by several methods that include hybridization and biotin/streptavidin linkage. In some embodiments, a protein or aptamer variant is encapsulated and in vitro transcription and translation of DNA molecules or translation of RNA molecules coding for protein or polypeptide variants in microcompartments is performed. In some embodiments, such encapsulation occurs in water in oil droplets, liposomes or microchambers. In some embodiments, using pM nucleic acid concentrations provides that most droplets contain either a single nucleic acid molecule or no molecule at all. Inside the microcompartments, nucleic acids and the corresponding translated polypeptides or proteins will form stable complexes that will maintain the genotype/phenotype linkage. This will happen because the polypeptides or proteins have a natural affinity for DNA (for example polymerases, helicases, DNA remodeling/binding proteins in general) or are fused to a domain having strong DNA affinity (for examples meganucleases, terminus site-binding (TER) proteins) or are fused to proteins engineered to interact covalently or non-covalently with a suitably modified nucleic acid. Intact complexes may be recovered from the microcompartments and coupled by hybridization, biotin/streptavidin linkage or otherwise to the slide. In some embodiments, nucleic acids, or one-to-one complexes of the nucleic acid and the polypeptide or protein it encodes, are deposited on a microscope slide enclosed in a microfluidic chamber. The slide can be engineered to improve the signal-to-noise ratio, for example by using micropatterning, plasmonic methods or zero-mode waveguides. The phenotype (for example binding or catalytic activity) of each nucleic acid or protein molecule may be characterized by single-molecule methods.

In some embodiments, phenotypes are mapped to genotypes, either by binding a polymerase to the glass slide coupled with a nucleic acid and directly reading its sequence or the sequence of an associated DNA barcode (e.g., by observing the incorporation of fluorescently labeled nucleotides (as in the SMRT sequencing by PacBio)), or by reading the barcode associated to the gene using single-molecule fluorescence sequencing-by-hybridization methods.

In some embodiments, this information is used to identify mutants with the desired properties and to design new libraries for the additional rounds of selection. The analysis process for identifying mutants with desired properties may include machine learning/deep learning or other Artificial Intelligence methods.

Another aspect of the present disclosure relates to identifying variant polymerases using the methods described herein. Polymerases have been in the focus of biotechnological research for decades. They are essential in various applications ranging from different forms of DNA amplification, RNA reverse-transcription, and, notably, DNA sequencing.

Many available DNA sequencing techniques, high- or low-throughput, rely on a specifically engineered polymerases. This is especially true for sequencing-by-synthesis methods where the template sequence is read by detecting fluorescently labeled nucleotides bound in the polymerase active site.

The present disclosure relates, in part, to a method of identifying nucleic acids coding for polymerases having desired properties, based on single-molecule measurements.

In some embodiments, libraries of RNAs or DNAs in which the sequence of each molecule may be encoded by a specific sequence barcode are created. In some embodiments, a random or designed library is created by random mutagenesis or synthetic gene synthesis. DNA barcodes may be appended to allow identification of variant genes by single-molecule sequencing.

In some embodiments, DNA (or translate RNA) molecules coding for polymerase variants in low-volume (e.g., fL, pL, μL) emulsion droplets produced by microfluidics are encapsulated and in vitro transcribed and translated. In some embodiments, using pM nucleic acid concentrations provides that most droplets contain either a single nucleic acid molecule or no molecule at all. Polymerases expressed in droplets have a natural affinity for nucleic acids and bind to their coding nucleic acids. Intact and stable complexes may be recovered from the emulsion and loaded in the wells of a suitably functionalized zero-mode waveguide (ZMW) or on a suitably functionalized glass slide.

In some embodiments, the incorporation of phospholinked dC, dA, dG and dT nucleotides coupled to different colored fluorophores is used to characterize polymerase phenotype by single-molecule methods. This approach allows the measurement of the lifetimes of the bright (nucleotide bound) and dark (free) states along the enzymatic pathways and of how these times depend on the sequence and on chemical modification patterns. Moreover, as the polymerase copies its own gene, the fluorescence output is directly used to read the template sequence, or the associated barcode, allowing genotype/phenotype mapping. This method provides, in some embodiments, a throughput of 10⁵ mutants/experiment on a zero-mode waveguide. In some embodiments, the polymerase is loaded on a magnetic tweezers instrument and the polymerization activity is observed by monitoring the extension of the template as it changes due to polymerization. This approach allows measurement of polymerization rate, processivity and pausing, and their dependence on the tension applied on the template. Following polymerization the sequence barcode can be read by mechanical sequencing.

In some embodiments, the information acquired in the screening is used to identify mutants with the desired properties and to design new libraries for the next round of selection. The analysis process to identify mutants with the desired properties may include machine learning/deep learning or other Artificial Intelligence methods.

Aspects of the disclosure relate to a method comprising: contacting an analyte with a first barcode recognition molecule and a second barcode recognition molecule, wherein the analyte is connected to a first nucleic acid index sequence and a second nucleic acid index sequence, wherein the first barcode recognition molecule specifically binds to the first nucleic acid index sequence and the second barcode recognition molecule specifically binds to the second nucleic acid index sequence; detecting a series of signal pulses indicative of binding interactions between (i) the first barcode recognition molecule and the first nucleic acid index sequence, and (ii) the second barcode recognition molecule and the second nucleic acid index sequence; and determining the identity of the analyte based on the series of signal pulses.

Aspects of the disclosure relate to a method comprising: (i) contacting an analyte with a first barcode recognition molecule and a second barcode recognition molecule, wherein the analyte is connected to a double-stranded barcode comprising a first nucleic acid index sequence and a second nucleic acid index sequence, wherein the first barcode recognition molecule specifically binds to the first nucleic acid index sequence and the second barcode recognition molecule specifically binds to the second nucleic acid index sequence; (ii) detecting a series of signal pulses indicative of binding interactions between (i) the first barcode recognition molecule and the first nucleic acid index sequence, and (ii) the second barcode recognition molecule and the second nucleic acid index sequence; and (iii) determining the identity of the analyte based on the series of signal pulses.

Aspects of the disclosure relate to a method comprising: (i) contacting an analyte with a first barcode recognition molecule, wherein the analyte is connected to a double-stranded barcode comprising a first nucleic acid index sequence and a second nucleic acid index sequence, wherein the first barcode recognition molecule specifically binds to the first nucleic acid index sequence and the second barcode recognition molecule specifically binds to the second nucleic acid index sequence; (ii) detecting a series of signal pulses indicative of binding interactions between (i) the first barcode recognition molecule and the first nucleic acid index sequence, and (ii) the first barcode recognition molecule and the second nucleic acid index sequence; and (iii) determining the identity of the analyte based on the series of signal pulses.

In some embodiments, prior to (i), the segments of the nucleic acid strands that are bound to the index sequences are removed from the double-stranded barcode, optionally wherein this contacting step is performed in the presence of single-stranded binding (SSB) protein. In some embodiments, prior to (i), the double-stranded barcode is contacted with a nicking enzyme to remove the nucleic acid strands that are bound to the index sequences, optionally wherein this contacting step is performed in the presence of single-stranded binding (SSB) protein.

Aspects of the disclosure relate to a method comprising: (i) attaching a biomolecule to a surface, wherein the biomolecule comprises (a) a protein variant and a molecular barcode comprising a first nucleic acid index sequence, or (b) an aptamer variant and a molecular barcode comprising a first nucleic acid index sequence; (ii) performing a phenotypic assay to determine one or more characteristics of the protein or aptamer variant; (iii) contacting the biomolecule with a first barcode recognition molecule, wherein the first barcode recognition molecule specifically binds to the first nucleic acid index sequence; (iv) detecting a series of signal pulses indicative of binding interactions between the first barcode recognition molecule and the first nucleic acid index sequence, and the second barcode recognition molecule and the second nucleic acid index sequence; (v) determining the identity of the biomolecule based on the series of signal pulses.

Aspects of the disclosure relate to a method comprising: (i) attaching a biomolecule to a surface, wherein the biomolecule comprises (a) a nucleic acid comprising a coding sequence encoding a protein variant and a molecular barcode comprising a first nucleic acid index sequence, and (b) the protein variant; (ii) performing a phenotypic assay to determine one or more characteristics of the protein variant; (iii) contacting the biomolecule with a first barcode recognition molecule, wherein the first barcode recognition molecule specifically binds to the first nucleic acid index sequence; (iv) detecting a series of signal pulses indicative of binding interactions between the first barcode recognition molecule and the first nucleic acid index sequence, and the second barcode recognition molecule and the second nucleic acid index sequence; (v) determining the identity of the biomolecule based on the series of signal pulses.

Aspects of the disclosure relate to a method of screening protein variants comprising: (i) generating a library of biomolecules, wherein each of the biomolecules comprises (a) a protein variant and a molecular barcode comprising an nucleic acid index sequence, or (b) an aptamer variant and a molecular barcode comprising an nucleic acid index sequence, wherein each of the biomolecules comprises a unique combination of nucleic acid index sequences; (ii) attaching each of the biomolecules to a surface; (iii) performing a phenotypic assay to determine one or more characteristics of each of the protein variants; (iv) contacting each of the biomolecules with a first barcode recognition molecule, wherein the first barcode recognition molecule specifically binds to the first nucleic acid index sequence; (v) detecting a series of signal pulses indicative of binding interactions between the first barcode recognition molecule and the first nucleic acid index sequence, and the second barcode recognition molecule and the second nucleic acid index sequence; (vi) determining the identity of each of the biomolecules based on the series of signal pulses; and (vii) identifying biomolecules comprising unique protein variants having one or more desired characteristics.

Aspects of the disclosure relate to a nucleic acid barcode comprising two, three, or four nucleic acid index sequences, wherein each of the nucleic acid index sequences is independently selected from any one of SEQ ID NOs: 25-28 or 36-59.

Aspects of the disclosure relate to a nucleic acid barcode comprising a first nucleic acid index sequence, a second nucleic acid index sequence, a third nucleic acid index sequence, and a fourth nucleic acid index sequence, wherein each of the nucleic acid index sequences comprises at least 6 nucleotides in length, optionally 6-9 nt, and wherein the second nucleic acid index sequence comprises one nucleobase substitution relative to the first nucleic acid index sequence, the third nucleic acid index sequence comprises two nucleobase substitutions relative to the first nucleic acid index sequence, and the fourth nucleic acid index sequence comprises three nucleobase substitutions relative to the first nucleic acid index sequence.

In some embodiments, the analyte is a DNA molecule, an RNA molecule, a polypeptide, a protein, or a nucleic acid aptamer. In some embodiments, the method or nucleic acid barcode further comprises a second index sequence, a third index sequence, and/or a fourth index sequence. In some embodiments, the analyte is a polymerase, an antibody, or a nucleic acid aptamer.

In some embodiments, the method or nucleic acid barcode further comprises identifying the analyte based on a known association between the molecular barcode and the analyte. In some embodiments, the signal pulse comprises a pulse duration that is characteristic of a dissociation rate of binding between the barcode recognition molecule and a site on the index. In some embodiments, at least one signal pulse is separated from another by an interpulse duration that is characteristic of an association rate of barcode recognition molecule binding.

In some embodiments, the barcode recognition molecule is an oligonucleotide, a nucleic acid, or a protein. In some embodiments, the barcode recognition molecule is a protein or a nucleic acid aptamer. In some embodiments, the barcode recognition molecule comprises a detectable label. In some embodiments, the detectable label is a luminescent label or a conductivity label. In some embodiments, the detectable label is a quenched label that is unquenched during binding between the barcode recognition molecule and the molecular barcode. In some embodiments, the detectable label is an unquenched label that is quenched during binding between the barcode recognition molecule and the index sequence.

In some embodiments, the series of signal pulses is a series of real-time signal pulses.

In some embodiments, the biomolecules are attached to a unique surface. In some embodiments, the biomolecules are attached to a well of a multi-well plate.

### Single-Molecule Kinetics

Aspects of the disclosure relate to identifying content of a molecular barcode. As used herein, “identifying,” “recognizing,” “recognition,” and like terms, in reference to a molecular barcode, includes determination of partial identity (e.g., partial sequence information) as well as full identity (e.g., full sequence information) of the molecular barcode. In some embodiments, the terminology includes determining or inferring a nucleotide sequence of at least a portion of a molecular barcode (e.g., based on complementarity with an oligonucleotide probe). In yet other embodiments, the terminology includes determining or inferring a certain characteristics of a molecular barcode, such as the presence or absence of a particular index sequence at one or more sites on a molecular barcode. Accordingly, in some embodiments, the terms “barcode content,” “barcode identity,” and like terms as used herein may refer to qualitative information pertaining to a molecular barcode and are not restricted to the specific sequence information (e.g., the nucleotide sequence of an index) that biochemically characterizes a molecular barcode.

In some embodiments, barcode recognition is performed by observing different association events between a barcode recognition molecule and a molecular barcode, where each association event produces a change in magnitude of a signal that persists for a duration of time. In some embodiments, these changes in magnitude are detected as a series of signal pulses, or a series of pulses in a signal trace output.

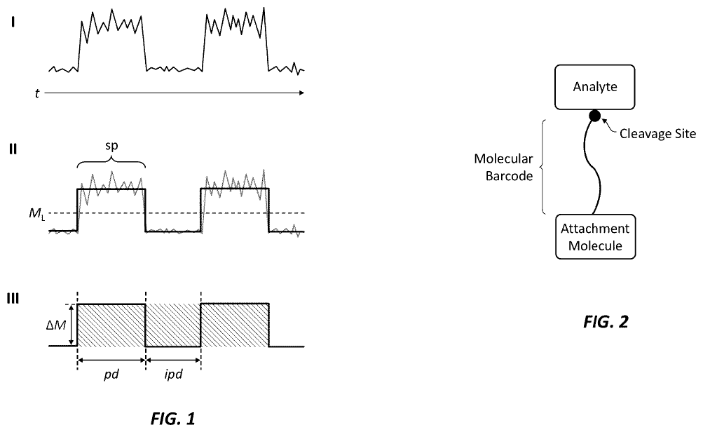

A non-limiting example of signal trace output and analysis is shown in FIG. 1. An example signal trace (I) is depicted with two signal pulses which each manifest as a peak in signal intensity that persists for a duration of time corresponding to an association event. Accordingly, the time duration between the two signal pulses having an approximately baseline signal may correspond to a duration of time during which a molecular barcode is not detectably associated with a barcode recognition molecule. In some embodiments, signal pulse data can be analyzed as illustrated in panels (II) and (III).

In some embodiments, signal data can be analyzed to extract signal pulse information by applying threshold levels to one or more parameters of the signal data. For example, panel (II) depicts a threshold magnitude level (“M_{L}”) applied to the signal data of the example signal trace (I). In some embodiments, M_{L} is a minimum difference between a signal detected at a point in time and a baseline determined for a given set of data. In some embodiments, a signal pulse (“sp”) is assigned to each portion of the data that is indicative of a change in magnitude exceeding M_{L} and persisting for a duration of time. In some embodiments, a threshold time duration may be applied to a portion of the data that satisfies M_{L} to determine whether a signal pulse is assigned to that portion. For example, experimental artifacts may give rise to a change in magnitude exceeding M_{L} that does not persist for a duration of time sufficient to assign a signal pulse with a desired confidence (e.g., non-specific detection events such as diffusion into an observation region or reagent sticking within an observation region). Accordingly, in some embodiments, a signal pulse is extracted from signal data based on a threshold magnitude level and a threshold time duration.

Extracted signal pulse information is shown in panel (II) with the example signal trace (I) superimposed for illustrative purposes. In some embodiments, a peak in magnitude of a signal pulse is determined by averaging the magnitude detected over a duration of time that persists above M_{L}. It should be appreciated that, in some embodiments, a “signal pulse” as used herein can refer to a change in signal data that persists for a duration of time above a baseline (e.g., raw signal data, as illustrated by the example signal trace (I)), or to signal pulse information extracted therefrom (e.g., processed signal data, as illustrated in panel (III)).

Panel (III) shows the signal pulse information extracted from the example signal trace (I). As shown, at least one signal pulse comprises a pulse duration (“pd”) corresponding to an association event between a barcode recognition molecule and a molecular barcode. In some embodiments, the pulse duration is characteristic of a dissociation rate of binding. Also as shown, at least one signal pulse is separated from another signal pulse by an interpulse duration (“ipd”). In some embodiments, the interpulse duration is characteristic of an association rate of binding. In some embodiments, a change in magnitude (“ΔM”) can be determined for a signal pulse based on a difference between baseline and the peak of a signal pulse.

In some embodiments, signal pulse information can be analyzed to identify barcode content based on a barcode-specific pattern in a series of signal pulses. For example, as shown in panel (III), in some embodiments, a barcode-specific pattern (shaded region) is determined based on pulse duration and interpulse duration. In some embodiments, a barcode-specific pattern is determined based on pulse duration, or a summary statistic for pulse duration as described elsewhere herein. In some embodiments, a barcode-specific pattern is determined based on any one or more of pulse duration, interpulse duration, and change in magnitude. In some embodiments, a barcode-specific pattern is determined to be associated with a particular feature and/or sequence of a molecular barcode (e.g., barcode content) based on reference data.

Accordingly, as illustrated by FIG. 1, in some embodiments, methods of the disclosure are performed by detecting a series of signal pulses indicative of association (e.g., binding) of a barcode recognition molecule with a molecular barcode. The series of signal pulses can be analyzed to determine a barcode-specific pattern in the series of signal pulses, and the barcode-specific pattern can be used to decipher barcode content.

In some embodiments, the series of signal pulses comprises a series of changes in magnitude of an optical signal over time. In some embodiments, the series of changes in the optical signal comprises a series of changes in luminescence produced during association events. In some embodiments, luminescence is produced by a detectable label associated with one or more reagents for barcode recognition. For example, in some embodiments, a barcode recognition molecule comprises a luminescent label. Examples of luminescent labels and their use in accordance with the disclosure are provided elsewhere herein.

In some embodiments, the series of signal pulses comprises a series of changes in magnitude of an electrical signal over time. In some embodiments, the series of changes in the electrical signal comprises a series of changes in conductance produced during association events. In some embodiments, conductivity is produced by a detectable label associated with one or more reagents for barcode recognition. For example, in some embodiments, a barcode recognition molecule comprises a conductivity label. Methods for identifying single molecules using conductivity labels have been described (see, e.g., U.S. Patent Publication No. 2017/0037462).

As described herein, signal pulse information may be used to identify barcode content based on a barcode-specific pattern in a series of signal pulses. In some embodiments, a barcode-specific pattern comprises a plurality of signal pulses, at least one signal pulse comprising a pulse duration. In some embodiments, the plurality of signal pulses may be characterized by a summary statistic (e.g., mean, median, time decay constant) of the distribution of pulse durations in a barcode-specific pattern. In some embodiments, the mean pulse duration of a barcode-specific pattern is between about 1 millisecond and about 10 seconds (e.g., between about 1 ms and about 1 s, between about 1 ms and about 100 ms, between about 1 ms and about ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different barcode-specific patterns corresponding to different barcode content may be distinguished from one another based on a statistically significant difference in the summary statistic. For example, in some embodiments, one barcode-specific pattern may be distinguishable from another barcode-specific pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one barcode-specific pattern is different from the mean pulse duration of another barcode-specific pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different barcode-specific patterns may require a greater number of pulse durations within each barcode-specific pattern to distinguish one from another with statistical confidence.

In some embodiments, a barcode-specific pattern generally refers to a plurality of association (e.g., binding) events between a barcode recognition molecule and a molecular barcode. In some embodiments, a barcode-specific pattern comprises at least 10 association events (e.g., at least 25, at least 50, at least 75, at least 100, at least 250, at least 500, at least 1,000, or more, association events). In some embodiments, a barcode-specific pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a barcode-specific pattern refers to a plurality of signal pulses which may be characterized by a summary statistic as described herein. In some embodiments, a barcode-specific pattern comprises at least 10 signal pulses (e.g., at least 25, at least 50, at least at least 100, at least 250, at least 500, at least 1,000, or more, signal pulses). In some embodiments, a barcode-specific pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a barcode-specific pattern refers to a plurality of association (e.g., binding) events between a barcode recognition molecule and a molecular barcode occurring over a time interval. In some embodiments, barcode recognition may be carried out by iterative wash cycles in which molecular barcodes are exposed to different sets of barcode recognition molecules over different time durations.

In some embodiments, experimental conditions can be configured to achieve a time interval that allows for sufficient association events which provide a desired confidence level with a barcode-specific pattern (e.g., before a given set of barcode recognition molecules is removed during wash cycles). This can be achieved, for example, by configuring the reaction conditions based on various properties, including: reagent concentration, molar ratio of one reagent to another (e.g., ratio of barcode recognition molecule to molecular barcode, ratio of one barcode recognition molecule to another), number of different reagent types (e.g., the number of different types of barcode recognition molecules), binding properties (e.g., kinetic and/or thermodynamic binding parameters for barcode recognition molecule binding), reagent modification (e.g., polyol and other protein modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkage groups), surface modification (e.g., modification of sample well surface, including molecular barcode immobilization), sample preparation (e.g., analyte size, molecular barcode modification for immobilization), and other aspects described herein.

### Molecular Barcodes

In some embodiments, methods provided herein comprise contacting a molecular barcode with one or more barcode recognition molecules that binds (e.g., specifically binds) to one or more nucleic acid index sequences on the molecular barcode. In some embodiments, a single barcode recognition molecule binds to the one or more nucleic acid index sequences. In some embodiments, a first barcode recognition molecule specifically binds to a first nucleic acid index sequence and a second barcode recognition molecule specifically binds to a second nucleic acid index sequence.

Accordingly, in some embodiments, a barcode recognition molecule can be used to decipher barcode content from a plurality of different single molecules in a mixture (e.g., different analytes comprising the same or different molecular barcodes). As an illustrative and non-limiting example, a multiplexed mixture can include a plurality of analytes attached to molecular barcodes. Some of these molecular barcodes can include a sample barcoded sequence that is indicative of sample origin for the analyte attached thereto, and a barcode recognition molecule that binds the sample barcoded sequence can be used to determine which analytes originated from the corresponding sample.

A molecular barcode may be a nucleic acid barcode. In some embodiments, a nucleic acid barcode is a single-stranded nucleic acid barcode. In other embodiments, a nucleic acid barcode is a double-stranded nucleic acid barcode. In some embodiments, a nucleic acid barcode comprises single-stranded segments and double-stranded segments.

A nucleic acid barcode may comprise DNA and/or RNA nucleotides. In some embodiments, a nucleic acid barcode comprises non-nucleic acid elements (e.g., polyethylene glycol spacers). A nucleic acid barcode may be any reasonable length. In some embodiments, a nucleic acid barcode comprises a length of 10-500 nucleotides in length. In some embodiments, a nucleic acid barcode comprises a length of 10-50 nucleotides, 10-100, 10-200, 10-250, 25-100, 50-150, 50-200, 100-200, 100-500, 200-400, 250-500, or 200-300 nucleotides in length.

A nucleic acid barcode of the disclosure generally comprises at least one nucleic acid index sequence. A nucleic acid index sequence is a sequence of nucleotides (e.g., RNA or DNA nucleotides) that can be detected or observed (e.g., through detection of transient binding interactions between the nucleic acid index sequence and one or more barcode recognition molecules) in order to assist with the identification of the nucleic acid barcode to which the index sequence belongs. In some embodiments, a nucleic acid barcode comprises 1, 2, 3, 4, 5, 6, 7, 8, 9, 10, or more nucleic acid index barcode sequences. A nucleic acid index sequence may comprise a length of 4-25 nucleotides, 4-20, 4-15, 5-10 nucleotides, 6-9 nucleotides, or 5-15 nucleotides. A nucleic acid index sequence may comprise a length of 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides.

In some embodiments, a nucleic acid barcode comprises a first nucleic acid index sequence. In some embodiments, a nucleic acid barcode comprises a first index nucleic acid sequence and a second nucleic acid index sequence. In some embodiments, a nucleic acid barcode comprises a first index nucleic acid sequence, a second nucleic acid index sequence, and a third nucleic acid index sequence. In some embodiments, a nucleic acid barcode comprises a first index nucleic acid sequence, a second nucleic acid index sequence, a third nucleic acid index sequence, and a fourth nucleic acid index sequence. In some embodiments, a first nucleic acid index sequence comprises a different sequence from a second, third, or fourth nucleic acid index sequence. In some embodiments, a first nucleic acid index sequence comprises the same sequence as a second, third, or fourth nucleic acid index sequence.

The nucleotide sequence of different nucleic acid index sequences within a nucleic acid barcode may comprise different, but similar sequences. For example, in some embodiments, a second nucleic acid index sequence comprises one nucleobase substitution relative to the nucleotide sequence of the first nucleic acid index sequence. In such an embodiment, the index sequences may comprise the same length and a one nucleobase substitution. Alternatively, the index sequences may comprise a different length but the same sequence, with the shorter index sequence lacking the 5′ or 3′ terminal nucleotide.

In some embodiments, a nucleic acid barcode comprises three, four, or more nucleic acid index sequences with each of the iterative index sequences comprising a single nucleotide difference between them. In some embodiments, a second nucleic acid index sequence, a third nucleic acid index sequence, and optionally a fourth nucleic acid index sequence may each comprise a one nucleotide substitution relative to a first nucleic acid index sequence, wherein each or some of the nucleotide substitution differences are distinct relative to the others.

Different nucleic acid index sequences may comprise different nucleotide sequences, different types of nucleotides (e.g., RNA vs DNA nucleotides), and/or different nucleic acid modifications (e.g., 2′ RNA modifications, internucleoside linkage modifications).

A nucleic acid barcode may comprise one or more spacers or linkers (e.g., positioned between two or more nucleic acid index sequences). Some nucleic acid barcodes of the disclosure comprise spacers between each of the distinct nucleic acid index sequences present in the barcode. A spacer may be a non-nucleic acid spacer. In some embodiments, a non-nucleic acid spacer is a sugar molecule, a polyethylene glycol spacer, a peptide spacer, or a small molecule spacer. In some embodiments, a spacer is a nucleic acid spacer.

A nucleic acid spacer may comprise a length of 5-35 nucleotides, 10-25 nucleotides, 15-30 nucleotides, 10-20 nucleotides, 10, 11, 12, 13, 14, 15, 16, 17, 18, 19, 20, or 25 nucleotides. A nucleic acid spacer can be single-stranded or double-stranded. In some embodiments, a nucleic acid spacer comprises DNA and/or RNA nucleotides.

A nucleic acid barcode (e.g., a double-stranded barcode) may comprise, in some embodiments, one or more sites within the barcode that allow for nicking or endonuclease activity. For example, in some embodiments, a nucleic acid barcode comprises one or more restriction sites that are specific for a nicking enzyme or endonuclease (e.g., a nicking endonuclease). The one or more restriction sites may surround a nucleic acid index sequence (e.g., a first restriction site is present at the 5′ end of the index sequence and a second restriction site is present at the 3′ end of the index sequence). In some embodiments, a nucleic acid barcode comprises a first nucleic acid index sequence and a second nucleic acid index sequence, wherein the two index sequences are separated by a spacer (e.g., a non-nucleic acid spacer or a nucleic acid spacer).

A nucleic acid barcode may comprise a first nucleic acid index sequence, a second nucleic acid index sequence, a third nucleic acid index sequence, and a fourth nucleic acid index sequence.

In some embodiments, a single-molecule construct for use in the methods of the disclosure may be of a general form as shown in FIG. 2. In some embodiments, the single-molecule construct includes a molecular barcode (e.g., kinetic barcode). In some embodiments, a molecular barcode of the disclosure is a nucleic acid barcode (e.g., a single-stranded nucleic acid). In some embodiments, a nucleic acid barcode comprises DNA, RNA, PNA, and/or LNA. In some embodiments, a molecular barcode is a polypeptide barcode.

As further shown in FIG. 2, in some embodiments, a molecular barcode is attached to an analyte (e.g., a payload molecule, a detector molecule). In some embodiments, an analyte is derived from a biological or synthetic source. In some embodiments, an analyte is derived from a serum sample, a blood sample, a tissue sample, or a single cell. In some embodiments, an analyte is a biomolecule. In some embodiments, an analyte is a nucleic acid or a polypeptide. In some embodiments, an analyte is a nucleic acid aptamer, a protein, or a protein fragment. In some embodiments, an analyte is a small molecule, a metabolite, or an antibody. In some embodiments, a molecular barcode is attached to an analyte via a linker. In some embodiments, the linker comprises a cleavage site (e.g., a photocleavable site). Accordingly, in some embodiments, a single-molecule construct comprising a cleavage sequence would allow for the removal of the analyte to simplify loading and/or analysis on a substrate surface (e.g., a chip).

Also as shown in FIG. 2, in some embodiments, a molecular barcode comprises an attachment molecule. In some embodiments, an attachment molecule is any moiety or linkage group suitable for surface immobilization of the molecular barcode. In some embodiments, the attachment molecule comprises a covalent or non-covalent linkage group. In some embodiments, the attachment molecule comprises a biotin moiety. In some embodiments, the attachment molecule comprises a bis-biotin moiety. Linkage groups and other compositions and methods useful for surface immobilization are described in further detail elsewhere herein and are known in the art.

It should be appreciated that FIG. 2 provides but one example configuration and is non-limiting with respect to single-molecule constructs of the disclosure. For example, in some embodiments, a cleavage site is an optional component which may not be incorporated into a single-molecule construct depending on a desired implementation. In some embodiments, again referring to FIG. 2, an attachment molecule can be adjacent to an analyte, such that a molecular barcode may be attached to a surface through the analyte. Examples of other configurations of single-molecule constructs and linkage strategies are provided elsewhere herein.

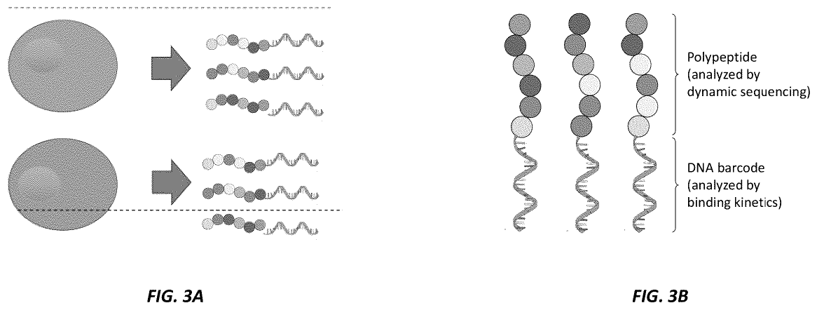

In some aspects, methods of the disclosure relate to a barcode deconvolution approach that involves deciphering molecular identity, sample origin, and/or location of a single molecule on an array. In some embodiments, methods provided herein are advantageously used to deconvolute molecular barcode information in a multiplexed sample. For example, methods of the disclosure can be applied to techniques for single-cell polypeptide sequencing. FIG. 3A shows a general process in which polypeptide molecules from single cells are labeled with cell-specific barcodes. In some embodiments, the resulting single-molecule constructs can be analyzed by polypeptide sequencing (e.g., dynamic peptide sequencing) and barcode recognition in accordance with the disclosure (FIG. 3B).

### Barcode Recognition Molecules

In some aspects, the disclosure provides barcode recognition molecules and methods of using the same. In some embodiments, a barcode recognition molecule can be selected or engineered based on desired binding kinetics with respect to a barcode site. For example, in some aspects, methods described herein can be performed in a multiplexed format in which a plurality of sites must be distinguished from one another based on binding interactions at each site. As such, the binding interactions at one site should be sufficiently different from binding interactions at another site, such that the different sites can be distinguished with higher confidence based on signal pulse information.

Without wishing to be bound by theory, a barcode recognition molecule binds to a barcode site according to a binding affinity (K_{D}) defined by an association rate, or an “on” rate, of binding (kₒₙ) and a dissociation rate, or an “off” rate, of binding (k_{off}). The rate constants k_{off} and kₒₙ are the similar determinants of pulse duration (e.g., the time corresponding to a detectable association event) and interpulse duration (e.g., the time between detectable association events), respectively. In some embodiments, these kinetic rate constants can be engineered to achieve pulse durations and pulse rates (e.g., the frequency of signal pulses) that give the best accuracy.

In some embodiments, an analyte (e.g., a nucleic acid, a peptide, a protein, a nucleic acid aptamer) is attached to a molecular barcode comprising 1, 2, 3 or 4 nucleic acid index sequences. In some embodiments, the analyte is contacted with a barcode recognition molecule that specifically binds one or more nucleic acid index sequences of the molecular barcode. In some embodiments, the barcode recognition molecule is an oligonucleotide. In some embodiments, the barcode recognition molecule is a protein or peptide. In some embodiments, the barcode recognition molecule comprises one or more detectable labels. In some embodiments, the barcode recognition molecule comprises one detectable label. In some embodiments, the binding of the barcode recognition molecule to the one or more nucleic acid index sequences generates a detectable signal. In some embodiments, the binding of the barcode recognition molecule to one or more nucleic acid index sequences generates a detectable signal that is indicative of the identity of the nucleic acid index sequences.

In some embodiments, the analyte is connected to a molecular barcode comprising one or more nucleic acid index sequences. In some embodiments, the analyte is contacted with a first barcode recognition molecule and a second barcode recognition molecule. In some embodiments, the first barcode recognition molecule specifically binds to a first nucleic acid index sequence of the molecular barcode. In some embodiments, the second barcode recognition molecule specifically binds to a second nucleic acid index sequence.

In some embodiments, the first barcode recognition molecule specifically binds to a first nucleic acid index sequence. In some embodiments, the first barcode recognition molecule specifically binds to a second nucleic acid index sequence. In some embodiments, the first barcode recognition molecule specifically binds to a third nucleic acid index sequence. In some embodiments, the first barcode recognition molecule specifically binds to a fourth nucleic acid index sequence. In some embodiments, the second barcode recognition molecule specifically binds to a first nucleic acid index sequence. In some embodiments, the second barcode recognition molecule specifically binds to a second nucleic acid index sequence. In some embodiments, the second barcode recognition molecule specifically binds to a third nucleic acid index sequence. In some embodiments, the second barcode recognition molecule specifically binds to a fourth nucleic acid index sequence. In some embodiments, a third barcode recognition molecule specifically binds to a first nucleic acid index sequence. In some embodiments, the third barcode recognition molecule specifically binds to a second nucleic acid index sequence. In some embodiments, the third barcode recognition molecule specifically binds to a third nucleic acid index sequence. In some embodiments, the third barcode recognition molecule specifically binds to a fourth nucleic acid index sequence. In some embodiments, a fourth barcode recognition molecule specifically binds to a first nucleic acid index sequence. In some embodiments, the fourth barcode recognition molecule specifically binds to a second nucleic acid index sequence. In some embodiments, the fourth barcode recognition molecule specifically binds to a third nucleic acid index sequence. In some embodiments, the fourth barcode recognition molecule specifically binds to a fourth nucleic acid index sequence. The binding characteristics (e.g., residence time, fluorescence emission, spectral properties) for each of the interactions between the different barcode recognition molecules and nucleic acid index sequences are, in some embodiments, unique and discrete for each interaction within a set of interactions.

In some embodiments, a barcode recognition molecule may be engineered by one skilled in the art using conventionally known techniques. In some embodiments, desirable properties may include an ability to bind with low to moderate affinity (e.g., with a K_{D} of about 50 nM or higher, for example, between about 50 nM and about 50 μM, between about 100 nM and about 10 μM, between about 500 nM and about 50 μM) to one or more sites on a molecular barcode. For example, in some aspects, the disclosure provides methods of barcode recognition by detecting reversible binding interactions, and barcode recognition molecules that reversibly bind molecular barcodes with low to moderate affinity advantageously provide more informative binding data and with higher certainty than high affinity binding interactions.

In some embodiments, a barcode recognition molecule binds one or more sites on a molecular barcode with a dissociation constant (K_{D}) of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M) without significantly binding to other off-target (e.g., non-complementary) sites. In some embodiments, a barcode recognition molecule binds one or more sites on a molecular barcode with a K_{D} of less than about 100 nM, less than about 50 nM, less than about 25 nM, less than about 10 nM, or less than about 1 nM. In some embodiments, a barcode recognition molecule binds one or more sites on a molecular barcode with a K_{D} of between about 50 nM and about 50 μM (e.g., between about 50 nM and about 500 nM, between about 50 nM and about 5 μM, between about 500 nM and about 50 μM, between about 5 μM and about 50 μM, or between about 10 μM and about 50 μM). In some embodiments, a barcode recognition molecule binds one or more sites on a molecular barcode with a K_{D} of about 50 nM.

In some embodiments, a barcode recognition molecule binds one or more sites on a molecular barcode with a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹.

In some embodiments, the value for K_{D} or k_{off} can be a known literature value, or the value can be determined empirically. For example, the value for K_{D} or k_{off} can be measured in a single-molecule assay or an ensemble assay. In some embodiments, the value for k_{off} can be determined empirically based on signal pulse information obtained in a single-molecule assay as described elsewhere herein. For example, the value for k_{off} can be approximated by the reciprocal of the mean pulse duration. In some embodiments, a barcode recognition molecule binds two or more chemically different barcode sites with a different K_{D} or k_{off} for each of the two or more sites. In some embodiments, a first K_{D} or k_{off} for a first site differs from a second K_{D} or k_{off} for a second site by at least 10% (e.g., at least 25%, at least 50%, at least 100%, or more). In some embodiments, the first and second values for K_{D} or k_{off} differ by about 10-25%, 25-50%, 50-75%, 75-100%, or more than 100%, for example by about 2-fold, 3-fold, 4-fold, 5-fold, or more.

As described herein, a barcode recognition molecule may be any biomolecule capable of binding one or more sites on a molecular barcode over other barcode sites. Recognition molecules include, for example, oligonucleotides, nucleic acids, and proteins, any of which may be synthetic or recombinant.

In some embodiments, a barcode recognition molecule is an oligonucleotide (e.g., an oligonucleotide probe). In some embodiments, methods provided herein can be performed by contacting a nucleic acid barcode with an oligonucleotide probe that binds one or more sites on the nucleic acid barcode. In some embodiments, the binding between the oligonucleotide probe and the nucleic acid barcode occurs via hybridization or annealing. Beyond certain experimental conditions (e.g., concentration, temperature), binding properties are in large part driven by length and content of the oligonucleotide probe and its degree of complementarity with the site on the nucleic acid barcode to which it binds (e.g., hybridizes or anneals). Accordingly, in some embodiments, oligonucleotide probes provide a variety of tunable features for modulating signal pulse characteristics, including, without limitation, length, nucleotide content (e.g., G/C content, nucleotide analogs with different binding characteristics, such as LNA or PNA analogs), degree of complementarity, and experimental factors, such as concentration, temperature, buffer conditions (e.g., pH, salt, magnesium), and DNA denaturing or stabilizing solvents.

In some embodiments, an oligonucleotide probe is at least four nucleotides in length. In some embodiments, an oligonucleotide probe is at least 5, at least 6, at least 7, at least 8, at least 9, at least 10, at least 12, at least 15, at least 20, at least 25, or at least 30 nucleotides in length. In some embodiments, an oligonucleotide probe is fewer than 30 nucleotides in length (e.g., fewer than 25, fewer than 20, fewer than 15, fewer than 12, fewer than 10 nucleotides in length). In some embodiments, an oligonucleotide probe is between about 3 and about 30 nucleotides in length (e.g., between about 3 and about 10, between about 3 and about 8, between about 5 and about 25, between about 5 and about 15, or between about 5 and 10 nucleotides in length). In some embodiments, an oligonucleotide probe is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, the oligonucleotide probe is 2-50, 3-49, 4-48, 5-47, 6-46, 7-45, 8-44, 9-43, 10-42, 11-41, 12-40, 13-39, 14-38, 15-37, 16-36, 17-35, 18-34, 19-33, 20-32, 21-31, 22-30, 23-29, 24-28, or 26-27 nucleotides in length. In some embodiments, the oligonucleotide probe is 4-15 nucleotides in length. In some embodiments, the oligonucleotide probe is 5-10 nucleotides in length. In some embodiments, the oligonucleotide probe is 6-9 nucleotides in length. In some embodiments, the oligonucleotide probe is 6 nucleotides in length. In some embodiments, the oligonucleotide probe is 7 nucleotides in length. In some embodiments, the oligonucleotide probe is 8 nucleotides in length. In some embodiments, the oligonucleotide probe is 9 nucleotides in length.

In some embodiments, an oligonucleotide probe can bind to, and provide barcode content information for, one or more barcode sites that are not fully complementary with the oligonucleotide probe. For example, in some embodiments, an oligonucleotide probe binds to one or more barcode sites having a sequence that is less than 100% complementary with the oligonucleotide (e.g., less than 99%, less than 98%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less).

In addition to oligonucleotides, nucleic acid aptamers can be used as barcode recognition molecules in accordance with the disclosure. Nucleic acid aptamers are nucleic acid molecules that have been engineered to bind targets with a desired affinity and selectivity. Accordingly, nucleic acid aptamers may be engineered to bind to a desired barcode site using selection and/or enrichment techniques known in the art. In some embodiments, a barcode recognition molecule comprises a nucleic acid aptamer, such as a DNA aptamer or an RNA aptamer.

In some embodiments, a barcode recognition molecule is a protein or polypeptide. In some embodiments, a barcode recognition molecule is an antibody or an antigen-binding portion of an antibody, an SH2 domain-containing protein or fragment thereof, or an inactivated enzymatic biomolecule, such as a peptidase, an aminotransferase, a ribozyme, an aptazyme, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING.”

In some embodiments, a barcode recognition molecule is an amino acid recognition molecule. For example, in some embodiments, a molecular barcode comprises a polypeptide barcode, and an amino acid recognition molecule can be used to decipher barcode content from the polypeptide. In some embodiments, an amino acid recognition molecule binds one or more types of terminal amino acids with different kinetic binding properties. In some embodiments, an amino acid recognition molecule binds different segments of a polypeptide with different kinetic binding properties. For example, in some embodiments, an amino acid recognition molecule binds to polypeptide segments comprising the same type of amino acid at the N-terminus or C-terminus but differing in amino acid content at the penultimate (e.g., n+1) and/or subsequent positions (e.g., different amino acid types at one or more of the second, third, fourth, fifth, or higher, position) relative to the terminal amino acid. These concepts (e.g., differential binding kinetics based on differences in amino acid content only at the penultimate position or higher) and additional examples of amino acid recognition molecules are described more fully in PCT International Publication No. WO2020102741A1, filed Nov. 15, 2019, titled “METHODS AND COMPOSITIONS FOR PROTEIN SEQUENCING,” which is incorporated by reference in its entirety.

In some embodiments, methods provided herein comprise contacting a molecular barcode with one or more barcode recognition molecules. For the purposes of this discussion, one or more barcode recognition molecules in the context of a method described herein may be alternatively referred to as a set of barcode recognition molecules. In some embodiments, a set of barcode recognition molecules comprises at least two and up to twenty (e.g., between 2 and 15, between 2 and 10, between 5 and 10, between 10 and 20) barcode recognition molecules. In some embodiments, a set of barcode recognition molecules comprises more than twenty (e.g., 20 to 25, 20 to 30) barcode recognition molecules. It should be appreciated, however, that any number of barcode recognition molecules may be used in accordance with a method of the disclosure to accommodate a desired use.

In accordance with the disclosure, in some embodiments, molecular barcode content can be identified by detecting luminescence from a label attached to a barcode recognition molecule. In some embodiments, a labeled barcode recognition molecule comprises a barcode recognition molecule that binds at least one molecular barcode and a luminescent label having a luminescence that is associated with the barcode recognition molecule. In this way, the luminescence (e.g., luminescence lifetime, luminescence intensity, and other luminescence properties described elsewhere herein, including luminescence-based kinetic binding data) may be associated with the binding of the barcode recognition molecule to identify the at least one molecular barcode. In some embodiments, a plurality of types of labeled barcode recognition molecules may be used in a method according to the disclosure, wherein each type comprises a luminescent label having a luminescence that is uniquely identifiable from among the plurality. Suitable luminescent labels may include luminescent molecules, such as fluorophore dyes, and are described elsewhere herein.

In some embodiments, at least one barcode recognition molecule comprises at least one detectable label. In some embodiments, a barcode recognition molecule comprises 1, 2, 3 or 4 detectable labels. In some embodiments, a barcode recognition molecule comprises two detectable labels. In some embodiments, a barcode recognition molecule comprises three detectable labels. In some embodiments a barcode recognition molecule comprises four detectable labels.

In some embodiments, the detectable label is a luminescent label. In some embodiments, the detectable label is a FRET label. In some embodiments, the detectable label is a lanthanide label. In some embodiments, the detectable label is a fluorescent label. In some embodiments, the detectable label is a conductivity label. In some embodiments, the detectable label is a fluorophore. In some embodiments, the detectable label is a dye. In some embodiments, the first barcode recognition molecule, the second barcode recognition molecule, the third barcode recognition molecule, and the fourth barcode recognition molecule each comprise different detectable labels. In some embodiments, the first barcode recognition molecule, the second barcode recognition molecule, the third barcode recognition molecule, and the fourth barcode recognition molecule comprise the same detectable labels. In some embodiments, the first barcode recognition molecule and the second barcode recognition molecule each comprise different detectable labels. In some embodiments, the first barcode recognition molecule and the second barcode recognition molecule comprise the same detectable label. In some embodiments, the detectable labels of the first barcode recognition molecule and the second barcode recognition molecule are fluorophores. In some embodiments, the fluorophore of the first barcode recognition molecule and the fluorophore of the second barcode recognition molecule comprise different absorption/emission spectral properties. In some embodiments, the fluorophore of the first barcode recognition molecule and the fluorophore of the second barcode recognition molecule produce different colors. In some embodiments, the detectable labels of the first, second, third, and fourth barcode recognition molecules are fluorophores. In some embodiments, the fluorophores of the first, second, third, and fourth barcode recognition molecules comprise different absorption/emission spectral properties. In some embodiments, the fluorophores of the first, second, third, and fourth barcode recognition molecules produce different colors. In some embodiments, at least one of the barcode recognition molecules comprises a quencher molecule. In some embodiments, the quencher molecule quenches the signal from the detectable label when the barcode recognition molecule is not bound to a nucleic acid index sequence, but does not quench the signal from the detectable label when the barcode recognition molecule is bound to a nucleic acid index sequence.

In some embodiments, the detectable label is only detectable when the barcode recognition molecule is bound to one or more indexes. In some embodiments, Total Internal Reflection Fluorescence (TIRF) illumination is used to detect the detectable label. In some embodiments, TIRF illumination allows a fluorescence signal to be detected only when the barcode recognition molecule is bound to a nucleic acid index sequence. In some embodiments, the detectable label is measured as luminescent intensity over time for each barcode.

In some embodiments, a barcode recognition molecule comprises a label having binding-induced luminescence. For example, in some embodiments, a labeled aptamer can comprise a donor label and an acceptor label. As a free and unbound molecule, the labeled aptamer adopts a conformation in which the donor and acceptor labels are separated by a distance that limits detectable FRET between the labels (e.g., about 10 nm or more). Upon binding to a barcode site, the labeled aptamer adopts a conformation in which the donor and acceptor labels are within a distance that promotes detectable FRET between the labels (e.g., about 10 nm or less). In yet other embodiments, a labeled aptamer can comprise a quenching moiety and function analogously to a molecular beacon, wherein luminescence is internally quenched as a free molecule and restored upon binding to a barcode site (see, e.g., Hamaguchi, et al. (2001) Analytical Biochemistry 294, 126-131). Similar and alternative labeling strategies would be apparent to those skilled in the art, such as the use of FRET between a labeled aptamer and a labeled molecular barcode. Without wishing to be bound by theory, it is thought that these and other types of mechanisms for binding-induced luminescence may advantageously reduce or eliminate background luminescence to increase overall sensitivity and accuracy of the methods described herein.

In some embodiments, molecular barcode content can be identified by detecting one or more electrical characteristics of a labeled barcode recognition molecule. In some embodiments, a labeled barcode recognition molecule comprises a barcode recognition molecule that binds at least one molecular barcode and a conductivity label that is associated with the barcode recognition molecule. In this way, the one or more electrical characteristics (e.g., charge, current oscillation color, and other electrical characteristics, including conductivity-based kinetic binding data) may be associated with the binding of the barcode recognition molecule to identify the at least one molecular barcode. In some embodiments, a plurality of types of labeled barcode recognition molecules may be used in a method according to the disclosure, wherein each type comprises a conductivity label that produces a change in an electrical signal (e.g., a change in conductance, such as a change in amplitude of conductivity and conductivity transitions of a barcode-specific pattern) that is uniquely identifiable from among the plurality. In some embodiments, the plurality of types of labeled barcode recognition molecules each comprises a conductivity label having a different number of charged groups (e.g., a different number of negatively and/or positively charged groups). Accordingly, in some embodiments, a conductivity label is a charge label. Examples of charge labels include dendrimers, nanoparticles, nucleic acids and other polymers having multiple charged groups. In some embodiments, a conductivity label is uniquely identifiable by its net charge (e.g., a net positive charge or a net negative charge), by its charge density, and/or by its number of charged groups.

### Methods of Identifying an Analyte

Some aspects of the disclosure provide methods for identifying an analyte (e.g., a protein or a nucleic acid analyte). In some embodiments, a method for identifying an analyte comprises contacting an analyte with one or more barcode recognition molecules, wherein the analyte is connected to a barcode comprising one or more nucleic acid index sequences, wherein the one or more barcode recognition molecules specifically bind to at least one of the one more nucleic acid index sequences. Following the contacting step, which brings the index sequence(s) belonging to the barcode in proximity with the barcode recognition molecule(s), a series of signal pulses indicative of binding interactions between the barcode recognition molecule(s) and the nucleic acid index sequence(s) are detected, such that the identity of the barcode (and the attached analyte) can be determined based on the series of signal pulses. In the context of the methods of the disclosure, a barcode recognition molecule “specifically binds” to a nucleic acid index sequence if the barcode recognition molecule preferentially binds to the nucleic acid index sequence with a higher affinity than to other molecules with which it only has non-specific interactions. In some embodiments, a barcode recognition molecule specifically binds to a nucleic acid index sequence if said barcode recognition molecule is an oligonucleotide probe (or nucleic acid-based barcode recognition molecule) and the barcode recognition molecule is complementary to the nucleic acid index sequence.

In some embodiments, a method of identifying an analyte involves specific binding events between a single nucleic acid index sequence of a barcode and multiple barcode recognition molecules (e.g, barcodes recognition molecules having different lengths and/or differentially labeled).

In some embodiments, a method of identifying an analyte involves specific binding events between multiple nucleic acid index sequences (e.g., 2, 3, or 4 nucleic acid index sequences) of a barcode and a single barcode recognition molecule that is capable of binding to one or more of the nucleic acid index sequences.

In some embodiments, a method of identifying an analyte involves specific binding events between multiple nucleic acid index sequences (e.g., 2, 3, or 4 nucleic acid index sequences) of a barcode and two or more barcode recognition molecules, wherein the barcode recognition molecules have differential binding capabilities.

In some embodiments, a method of identifying an analyte comprises contacting an analyte with one or more barcode recognition molecules, wherein the analyte is connected to a double-stranded barcode comprising one or more nucleic acid index sequences, wherein the one or more barcode recognition molecules specifically bind to the one or more nucleic acid index sequences, detecting a series of signal pulses indicative of binding interactions and determining the identity of the analyte based on the series of signal pulses. In some embodiments, methods utilizing double-stranded barcodes comprise a barcode processing step (e.g., to make the index sequences available for binding interactions with a barcode recognition molecule). For example, one or more segments of the nucleic acid strand that is bound to index sequences of the double-stranded barcode may be removed from the double-stranded barcode to allow for the index sequences to interact with barcode recognition molecules. In some embodiments, the one or more segments of the nucleic acid strand that are bound to the index sequences are removed from the double-stranded barcode using incubation with enzymes or chemical means. In some embodiments, the enzymes that remove the one or more segments of the nucleic acid strand that are bound to the index sequences are nicking enzymes or endonucleases that bind to specific restriction sites on the nucleic acid barcode.

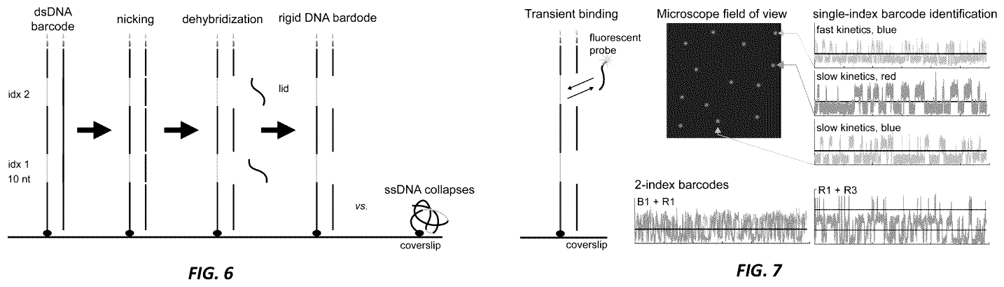

In some embodiments, such a step of removing one or more segments of the nucleic acid strand that is bound to index sequences of the double-stranded barcode is performed in the presence of single-stranded binding (SSB) protein. In some embodiments, the SSB protein is a herpes simplex virus (HSV-1) single-strand DNA-binding protein, a bacterial SSB, replication protein A, or Eukaryotic mitochondrial SSB. An example of such a processing step can be seen in FIG. 6.

An analyte may be attached to a surface using any means known to a person of ordinary skill in the art. A surface may be a glass, magnetic, or silica-based surface. In some embodiments, a surface is a surface of a well of a multi-well plate, optionally a 96-well plate or a 384-well plate. The analyte may be covalently or non-covalently attached to the surface.

The methods of the disclosure may be utilized to screen variants of an analyte. For example, in some embodiments, the methods of the disclosure may be used to screen protein variants or aptamer variants. A library of variants may be generated such that each variant within the library is attached to a unique barcode comprising a distinct series of nucleic acid index sequences as described herein.

In some embodiments, the screening methods of the disclosure allow for a coupling of phenotypic assays (e.g., binding assays or enzymatic assays) with the barcode determinations. This can allow for rapid identification of variants having one or more desired characteristics.

### Luminescent Labels

As used herein, a luminescent label is a molecule that absorbs one or more photons and may subsequently emit one or more photons after one or more time durations. In some embodiments, the term is used interchangeably with “label” or “luminescent molecule” depending on context. A luminescent label in accordance with certain embodiments described herein may refer to a luminescent label of a labeled recognition molecule (e.g., a labeled barcode recognition molecule), a luminescent label of a labeled peptidase (e.g., a labeled exopeptidase, a labeled non-specific exopeptidase), a luminescent label of a labeled peptide, a luminescent label of a labeled cofactor, or another labeled composition described herein. In some embodiments, a luminescent label in accordance with the disclosure refers to a labeled amino acid of a labeled polypeptide comprising one or more labeled amino acids.

In some embodiments, a luminescent label may comprise a first and second chromophore. In some embodiments, an excited state of the first chromophore is capable of relaxation via an energy transfer to the second chromophore. In some embodiments, the energy transfer is a Förster resonance energy transfer (FRET). Such a FRET pair may be useful for providing a luminescent label with properties that make the label easier to differentiate from amongst a plurality of luminescent labels in a mixture. In yet other embodiments, a FRET pair comprises a first chromophore of a first luminescent label and a second chromophore of a second luminescent label. In certain embodiments, the FRET pair may absorb excitation energy in a first spectral range and emit luminescence in a second spectral range. In general, a donor chromophore is selected that has a substantial spectrum of the acceptor chromophore. Furthermore, it may also be desirable in certain applications that the donor have an excitation maximum near a laser frequency such as Helium-Cadmium 442 nM, Argon 488 nM, NdrYAG 532 nm, He—Ne 633 nm, etc. In such applications, the use of intense laser light can serve as an effective means to excite the donor fluorophore.

In some embodiments, an amino acid recognition molecule of the disclosure comprises a detectable label that undergoes Forster/fluorescence resonance energy transfer (FRET). In some embodiments, amino acid sequence information can be determined by contacting a single polypeptide molecule with at least two amino acid binding proteins comprising different FRET labels. In some embodiments, the different FRET labels comprise different configurations of chromophores of the same type. In some embodiments, the different configurations permit different FRET efficiencies, such that the different FRET labels (and the different types of amino acids associated therewith) may be distinguishable by relative emission intensities of donor and acceptor chromophores.

In some embodiments, the disclosure provides compositions comprising two or more types of amino acid recognition molecules, where each type binds the same type of amino acid and comprises a different type of label. For example, in some embodiments, the composition comprises a first and second amino acid binding protein comprising a first and second label, respectively, where the first label is different from the second label, and where the first and second amino acid binding proteins binds the same type of amino acid. Such compositions can be used in polypeptide sequencing reactions to provide increased confidence levels in determining the identity of an amino acid of a polypeptide.

In some aspects, the disclosure provides a composition comprising: a first amino acid binding protein comprising a first FRET label, where the first FRET label has a first emission spectrum comprising peaks of a first wavelength and a second wavelength; and a second amino acid binding protein comprising a second FRET label, where the second FRET label has a second emission spectrum comprising peaks of the first wavelength and the second wavelength.

In some embodiments, emission intensities at one or both peaks of the first emission spectrum are different from emission intensities at one or both peaks of the second emission spectrum. In some embodiments, each peak is characterized by an emission intensity at a particular wavelength (e.g., the first or second wavelength), and the emission intensity at the particular wavelength in the first and second emission spectra are different. In some embodiments, emission intensities at the first and second wavelengths in the first emission spectrum are different from emission intensities at the first and second wavelengths in the second emission spectrum. For example, in some embodiments, emission intensity at the first wavelength in the first emission spectrum is different from emission intensity at the first wavelength in the second emission spectrum, and emission intensity at the second wavelength in the first emission spectrum is different from emission intensity at the second wavelength in the second emission spectrum.

In some embodiments, the first wavelength is an emission wavelength for a donor chromophore of each FRET label, and the second wavelength is an emission wavelength for an acceptor chromophore of each FRET label. In some embodiments, the ratio of the donor chromophore to the acceptor chromophore in each FRET label is 1:1, 2:1, 3:1, 4:1, 5:1, 1:2, 1:3, 1:4, or 1:5.

In some embodiments, the first FRET label has a first FRET efficiency, and the second FRET label has a second FRET efficiency, where the first FRET efficiency is different from the second FRET efficiency. In some embodiments, the first FRET efficiency differs from the second FRET efficiency by at least about 5%. In some embodiments, the first amino acid binding protein comprises the first FRET label in a first configuration that permits the first FRET efficiency; and the second amino acid binding protein comprises the second FRET label in a second configuration that permits the second FRET efficiency. In some embodiments, the first configuration maintains a first distance between chromophores in the first FRET label, and the second configuration maintains a second distance between the chromophores in the second FRET label, where the first distance is different from the second distance.

In some embodiments, the first amino acid binding protein is attached to the first FRET label through a first linkage group, and the second amino acid binding protein is attached to the second FRET label through a second linkage group. In some embodiments, chromophores of the first FRET label are attached to the first linkage group in the first configuration, and chromophores of the second FRET label are attached to the second linkage group in the second configuration.

In some embodiments, the first FRET label comprises a first chromophore, and the second FRET label comprises a second chromophore that is identical to the first chromophore. In some embodiments, the first FRET label comprises a first plurality of chromophores, and the second FRET label comprises a second plurality of chromophores, where chromophores of the first plurality are identical to chromophores of the second plurality.

In some embodiments, the composition further comprises at least one amino acid binding protein comprising a non-FRET label. In some embodiments, the non-FRET label comprises a fluorophore. In some embodiments, the non-FRET label comprises a chromophore identical to a donor or acceptor chromophore of the first FRET label.

In some embodiments, the first emission spectrum distinctly identifies a first type of amino acid, and the second emission spectrum distinctly identifies a second type of amino acid. In some embodiments, the first and second types of amino acids are naturally occurring amino acids of a different type. In some embodiments, the first amino acid binding protein binds to a first subset of types of amino acids, and the second amino acid binding protein binds to a second subset of types of amino acids. In some embodiments, the first subset of types of amino acids is different from the second subset of types of amino acids.

In some embodiments, the composition further comprises at least one peptidase. In some embodiments, the molar ratio of the first or second amino acid binding protein to the peptidase is between about 1:1,000 and about 1:1 or between about 1:1 and about 100:1. In some embodiments, the molar ratio of the first or second amino acid binding protein to the peptidase is between about 1:100 and about 1:1 or between about 1:1 and about 10:1. In some embodiments, the molar ratio of the first or second amino acid binding protein to the peptidase is about 1:1,000, about 1:500, about 1:200, about 1:100, about 1:10, about 1:5, about 1:2, about 1:1, about 5:1, about 10:1, about 50:1, about 100:1.

In some embodiments, the first and second amino acid binding proteins are each independently selected from a Gid protein, a UBR-box protein or UBR-box domain-containing fragment thereof, a p62 protein or ZZ domain-containing fragment thereof, and a ClpS protein. In some embodiments, at least one of the first and second amino acid binding proteins is a ClpS protein.

In some aspects, the disclosure provides a labeled amino acid recognition molecule comprising: a nucleic acid comprising a FRET label, where the FRET label has an emission spectrum comprising at least two peaks that distinctly identify a terminal amino acid; and at least one amino acid binding protein attached to the nucleic acid, where the nucleic acid forms a covalent or non-covalent linkage group between the at least one amino acid binding protein and the FRET label.

In some embodiments, the FRET label has a FRET efficiency of less than 90%. In some embodiments, the FRET label is attached to the nucleic acid in a configuration that permits the FRET efficiency. In some embodiments, the FRET label comprises a plurality of chromophores attached to a respective plurality of attachment sites on the nucleic acid. In some embodiments, each attachment site is separated by another attachment site of the plurality by between 5 and 100 nucleotide bases or nucleotide base pairs on the nucleic acid.

In some embodiments, the FRET label is attached to the nucleic acid through a biomolecule that forms a covalent or non-covalent linkage group between the FRET label and the nucleic acid. In some embodiments, the FRET label comprises a plurality of chromophores attached to a respective plurality of attachment sites on the biomolecule. In some embodiments, the biomolecule is a multivalent protein.

In some embodiments, the nucleic acid is a double-stranded nucleic acid comprising a first oligonucleotide strand hybridized with a second oligonucleotide strand. In some embodiments, the at least one amino acid binding protein is attached to the first oligonucleotide strand, where the FRET label is attached to the first oligonucleotide strand. In some embodiments, the at least one amino acid binding protein is attached to the first oligonucleotide strand, and where the FRET label is attached to the second oligonucleotide strand. In some embodiments, the at least one amino acid binding protein is attached to the first oligonucleotide strand, where chromophores of the FRET label are attached to each of the first and second oligonucleotide strands.

In some embodiments, the FRET label comprises a donor chromophore and an acceptor chromophore, where the ratio of the donor chromophore to the acceptor chromophore is 1:1, 2:1, 3:1, 4:1, 5:1, 1:2, 1:3, 1:4, or 1:5.

In some embodiments, an acceptor chromophore of a FRET label has a substantial overlap of its excitation spectrum with the emission spectrum of a donor chromophore of the FRET label. In some embodiments, the wavelength maximum of the emission spectrum of the acceptor chromophore is preferably at least 10 nm greater than the wavelength maximum of the excitation spectrum of the donor chromophore. Additional examples of useful FRET labels include, e.g., those described in U.S. Pat. Nos. 5,654,419, 5,688,648, 5,853,992, 5,863,727, 6,008,373, 6,150,107, 6,177,249, 6,335,440, 6,348, 596, 6,479,303, 6,545,164, 6,849,745, 6,696,255, and 6,908,769 and Published U.S. Patent Application Nos. 2002/0168641, 2003/0143594, and 2004/0076979, the disclosures of which are incorporated herein by reference for all purposes.

In some embodiments, a luminescent label refers to a fluorophore or a dye. Typically, a luminescent label comprises an aromatic or heteroaromatic compound and can be a pyrene, anthracene, naphthalene, naphthylamine, acridine, stilbene, indole, benzindole, oxazole, carbazole, thiazole, benzothiazole, benzoxazole, phenanthridine, phenoxazine, porphyrin, quinoline, ethidium, benzamide, cyanine, carbocyanine, salicylate, anthranilate, coumarin, fluoroscein, rhodamine, xanthene, or other like compound.

In some embodiments, a luminescent label comprises a dye selected from one or more of the following: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor® 405, Alexa Fluor® 430, Alexa Fluor® 480, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610-X, Alexa Fluor® 633, Alexa Fluor® 647, Alexa Fluor® 660, Alexa Fluor® 680, Alexa Fluor® 700, Alexa Fluor® 750, Alexa Fluor® 790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™350, CF™405M, CF™405S, CF™488A, CF™514, CF™532, CF™543, CF™546, CF™555, CF™568, CF™594, CF™620R, CF™633, CF™633-V1, CF™640R, CF™640R-V1, CF™640R-V2, CF™660C, CF™660R, CF™680, CF™680R, CF™680R-V1, CF™750, CF™770, CF™790, Chromeo™ 642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight® 350, DyLight® 405, DyLight® 415-Col, DyLight® 425Q, DyLight® 485-LS, DyLight® 488, DyLight® 504Q, DyLight® 510-LS, DyLight® 515-LS, DyLight® 521-LS, DyLight® 530-R2, DyLight® 543Q, DyLight® 550, DyLight® 554-R0, DyLight® 554-R1, DyLight® 590-R2, DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-05, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight® 730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight® 775-B4, DyLight® 780-B1, DyLight® 780-B2, DyLight® 780-B3, DyLight® 800, DyLight® 830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™, Pacific Green™, Pacific Orange™, PET, PF350, PF405, PF415, PF488, PF505, PF532, PF546, PF555P, PF568, PF594, PF610, PF633P, PF647P, Quasar® 570, Quasar® 670, Quasar® 705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™ 633, Seta™ 650, Seta™ 660, Seta™ 670, Seta™ 680, Seta™ 700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7.

### Luminescence

In some aspects, the disclosure relates to identification of analytes or biomolecules based on one or more luminescence properties of a luminescent label attached to a barcode recognition molecule. In some embodiments, a luminescent label is identified based on luminescence lifetime, luminescence intensity, brightness, absorption spectra, emission spectra, luminescence quantum yield, or a combination of two or more thereof. In some embodiments, a plurality of types of luminescent labels can be distinguished from each other based on different luminescence lifetimes, luminescence intensities, brightnesses, absorption spectra, emission spectra, luminescence quantum yields, or combinations of two or more thereof. In some embodiments, a luminescent label is identified based on luminescence intensity alone. Identifying may mean assigning the exact identity and/or quantity of one type of amino acid (e.g., a single type or a subset of types) associated with a luminescent label, and may also mean assigning an amino acid location in a polypeptide relative to other types of amino acids.

In some embodiments, luminescence is detected by exposing a luminescent label to a series of separate light pulses and evaluating the timing or other properties of each photon that is emitted from the label. In some embodiments, information for a plurality of photons emitted sequentially from a label is aggregated and evaluated to identify the label and thereby identify an associated type of amino acid. In some embodiments, a luminescence lifetime of a label is determined from a plurality of photons that are emitted sequentially from the label, and the luminescence lifetime can be used to identify the label. In some embodiments, a luminescence intensity of a label is determined from a plurality of photons that are emitted sequentially from the label, and the luminescence intensity can be used to identify the label. In some embodiments, a luminescence lifetime and luminescence intensity of a label is determined from a plurality of photons that are emitted sequentially from the label, and the luminescence lifetime and luminescence intensity can be used to identify the label.

In some aspects of the disclosure, a single polypeptide molecule is exposed to a plurality of separate light pulses and a series of emitted photons are detected and analyzed. In some embodiments, the series of emitted photons provides information about the single polypeptide molecule that is present and that does not change in the reaction sample over the time of the experiment. However, in some embodiments, the series of emitted photons provides information about a series of different molecules that are present at different times in the reaction sample (e.g., as a reaction or process progresses). By way of example and not limitation, such information may be used to sequence and/or identify a polypeptide subjected to chemical or enzymatic degradation in accordance with the disclosure.

In certain embodiments, a luminescent label absorbs one photon and emits one photon after a time duration. In some embodiments, the luminescence lifetime of a label can be determined or estimated by measuring the time duration. In some embodiments, the luminescence lifetime of a label can be determined or estimated by measuring a plurality of time durations for multiple pulse events and emission events. In some embodiments, the luminescence lifetime of a label can be differentiated amongst the luminescence lifetimes of a plurality of types of labels by measuring the time duration. In some embodiments, the luminescence lifetime of a label can be differentiated amongst the luminescence lifetimes of a plurality of types of labels by measuring a plurality of time durations for multiple pulse events and emission events. In certain embodiments, a label is identified or differentiated amongst a plurality of types of labels by determining or estimating the luminescence lifetime of the label. In certain embodiments, a label is identified or differentiated amongst a plurality of types of labels by differentiating the luminescence lifetime of the label amongst a plurality of the luminescence lifetimes of a plurality of types of labels.

Determination of a luminescence lifetime of a luminescent label can be performed using any suitable method (e.g., by measuring the lifetime using a suitable technique or by determining time-dependent characteristics of emission). In some embodiments, determining the luminescence lifetime of one label comprises determining the lifetime relative to another label. In some embodiments, determining the luminescence lifetime of a label comprises determining the lifetime relative to a reference. In some embodiments, determining the luminescence lifetime of a label comprises measuring the lifetime (e.g., fluorescence lifetime). In some embodiments, determining the luminescence lifetime of a label comprises determining one or more temporal characteristics that are indicative of lifetime. In some embodiments, the luminescence lifetime of a label can be determined based on a distribution of a plurality of emission events (e.g., 1, 2, 3, 4, 5, 6, 7, 8, 9, 10, 11, 12, 13, 14, 15, 16, 17, 18, 19, 20, 30, 40, 50, 60, 70, 80, 90, 100, or more emission events) occurring across one or more time-gated windows relative to an excitation pulse. For example, a luminescence lifetime of a label can be distinguished from a plurality of labels having different luminescence lifetimes based on the distribution of photon arrival times measured with respect to an excitation pulse.

It should be appreciated that a luminescence lifetime of a luminescent label is indicative of the timing of photons emitted after the label reaches an excited state and the label can be distinguished by information indicative of the timing of the photons. Some embodiments may include distinguishing a label from a plurality of labels based on the luminescence lifetime of the label by measuring times associated with photons emitted by the label. The distribution of times may provide an indication of the luminescence lifetime which may be determined from the distribution. In some embodiments, the label is distinguishable from the plurality of labels based on the distribution of times, such as by comparing the distribution of times to a reference distribution corresponding to a known label. In some embodiments, a value for the luminescence lifetime is determined from the distribution of times.

As used herein, in some embodiments, luminescence intensity refers to the number of emitted photons per unit time that are emitted by a luminescent label which is being excited by delivery of a pulsed excitation energy. In some embodiments, the luminescence intensity refers to the detected number of emitted photons per unit time that are emitted by a label which is being excited by delivery of a pulsed excitation energy, and are detected by a particular sensor or set of sensors. In some embodiments, the luminescence intensity of a label can be differentiated amongst the luminescence intensities of a plurality of types of labels (e.g., FRET labels). In some embodiments, a label is identified or differentiated amongst a plurality of types of labels by determining or estimating the luminescence intensity of the label. In some embodiments, a label is identified or differentiated amongst a plurality of types of labels by differentiating the luminescence intensity of the label amongst a plurality of the luminescence intensities of a plurality of types of labels.

As used herein, in some embodiments, brightness refers to a parameter that reports on the average emission intensity per luminescent label. Thus, in some embodiments, “emission intensity” may be used to generally refer to brightness of a composition comprising one or more labels. In some embodiments, brightness of a label is equal to the product of its quantum yield and extinction coefficient.

As used herein, in some embodiments, luminescence quantum yield refers to the fraction of excitation events at a given wavelength or within a given spectral range that lead to an emission event, and is typically less than 1. In some embodiments, the luminescence quantum yield of a luminescent label described herein is between 0 and about 0.001, between about 0.001 and about 0.01, between about 0.01 and about 0.1, between about 0.1 and about 0.5, between about 0.5 and 0.9, or between about 0.9 and 1. In some embodiments, a label is identified by determining or estimating the luminescence quantum yield.

As used herein, in some embodiments, an excitation energy is a pulse of light from a light source. In some embodiments, an excitation energy is in the visible spectrum. In some embodiments, an excitation energy is in the ultraviolet spectrum. In some embodiments, an excitation energy is in the infrared spectrum. In some embodiments, an excitation energy is at or near the absorption maximum of a luminescent label from which a plurality of emitted photons are to be detected. In certain embodiments, the excitation energy is between about 500 nm and about 700 nm (e.g., between about 500 nm and about 600 nm, between about 600 nm and about 700 nm, between about 500 nm and about 550 nm, between about 550 nm and about 600 nm, between about 600 nm and about 650 nm, or between about 650 nm and about 700 nm). In certain embodiments, an excitation energy may be monochromatic or confined to a spectral range. In some embodiments, a spectral range has a range of between about 0.1 nm and about 1 nm, between about 1 nm and about 2 nm, or between about 2 nm and about 5 nm. In some embodiments, a spectral range has a range of between about 5 nm and about 10 nm, between about 10 nm and about 50 nm, or between about 50 nm and about 100 nm.

### EXAMPLES

### Example 1. Molecular Barcode Design

This example relates to the design of molecular barcodes and characterization of index binding kinetics.

### Molecular Barcode Design

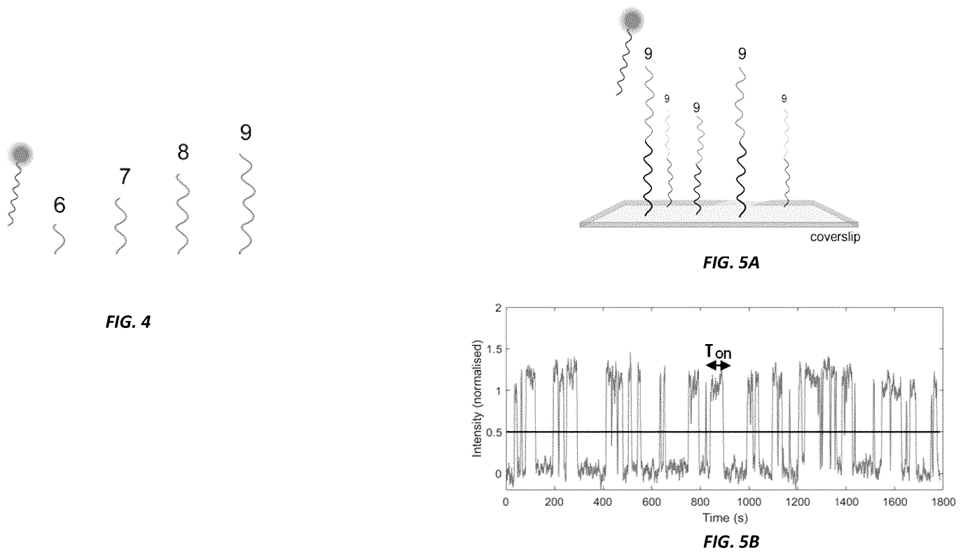

Six orthogonal index sequences were identified (A-F, Tables 1-6). These nucleic acid index sequences are adapted to specifically bind a 9-nucleotide (nt) fluorescently-labeled probe. The longest index for each sequence has a 9-nt complementarity with the probe. To create four different index lengths for each sequence (6-9 nt), nucleotides were removed from the 3′ end (FIG. 4). The probe can bind to all four index lengths; however, the residence times (binding kinetics) of the probe depend on the length of the sequence overlap, allowing for discrimination and identification of the four nucleic acid index sequences relative to one another.

### Characterization of Index Binding Kinetics

To characterize the binding kinetics (e.g., residence times) of fluorescently-labeled oligonucleotide probes binding to each of the indexes, single-stranded nucleic acid barcode sequences were used. The single-stranded barcode sequences contained a nucleic acid index sequence and a 15-T spacer (15 consecutive thymine nucleobases), and have a biotin on the 5′ end (Tables 1-6). The biotin facilitated tethering of the barcodes to a microscope coverslip (FIG. 5A). Using microfluidics, the fluorescently-labeled oligonucleotide probe can be introduced. The probe sequence is complementary to the nucleic acid index sequence. Because Total Internal Reflection Fluorescence (TIRE) illumination was used, a fluorescence signal was only detected when the probe is bound to the index. The probes bound transiently to the barcode index, resulting in a telegraph-like signal, which was measured as intensity over time for each barcode (FIG. 5B). This allows for the measurement of the average time the probe was bound to the index (tₒₙ or residence time). For Tables 1-6, the index sequences are in bold.

TABLE 1
Barcode A indexes

| | Full Barcode | Nucleic acid |
|---|---|---|
| | sequence | index sequence |
| 6mer | 5′-TTTTTTTTTTTTTTT | TGTTCC |
| | TGTTCC | (SEQ ID NO: 36) |
| | (SEQ ID NO: 1) | |
| 7mer | 5′-TTTTTTTTTTTTTTT | TGTTCCT |
| | TGTTCCT | (SEQ ID NO: 37) |
| | (SEQ ID NO: 2) | |
| 8mer | 5′-TTTTTTTTTTTTTTT | TGTTCCTC |
| | TGTTCCTC | (SEQ ID NO: 38) |
| | (SEQ ID NO: 3) | |
| 9mer | 5′-TTTTTTTTTTTTTTT | TGTTCCTCT |
| | TGTTCCTCT | (SEQ ID NO: 39) |
| | (SEQ ID NO: 4) | |
| Probe | 5′-AF647-AGA GGA ACA | |
| | (SEQ ID NO: 29) | |

TABLE 2
Barcode B indexes

| | Full Barcode | Nucleic acid |
|---|---|---|
| | sequence | index sequence |
| 6mer | 5′-TTTTTTTTTTTTTTT | CTCTCC |
| | CTCTCC | (SEQ ID NO: 40) |
| | (SEQ ID NO: 5) | |
| 7mer | 5′-TTTTTTTTTTTTTTT | CTCTCCC |
| | CTCTCCC | (SEQ ID NO: 41) |
| | (SEQ ID NO: 6) | |
| 8mer | 5′-TTTTTTTTTTTTTTT | CTCTCCCT |
| | CTCTCCCT | (SEQ ID NO: 42) |
| | (SEQ ID NO: 7) | |
| 9mer | 5′-TTTTTTTTTTTTT | CTCTCCCTC |
| | CTCTCCCTC | (SEQ ID NO: 43) |
| | (SEQ ID NO: 8) | |
| Probe | 5′-AF488-TT GAG GGA GAG | |
| | (SEQ ID NO: 30) | |

TABLE 3
Barcode C indexes

| | Full Barcode | Nucleic acid |
|---|---|---|
| | sequence | index sequence |
| 6mer | 5′-TTTTTTTTTTTTTTT | GCTTGT |
| | GCTTGT | (SEQ ID NO: 44) |
| | (SEQ ID NO: 9) | |
| 7mer | 5′-TTTTTTTTTTTTTTT | GCTTGTC |
| | GCTTGTC | (SEQ ID NO: 45) |
| | (SEQ ID NO: 10) | |
| 8mer | 5′-TTTTTTTTTTTTTTT | GCTTGTCT |
| | GCTTGTCT | (SEQ ID NO: 46) |
| | (SEQ ID NO: 11) | |
| 9mer | 5′-TTTTTTTTTTTTTTT | GCTTGTCTC |
| | GCTTGTCTC | (SEQ ID NO: 47) |
| | (SEQ ID NO: 12) | |
| Probe | 5′-AF594-TT GAG ACA AGC | |
| | (SEQ ID NO: 31) | |

TABLE 4
Barcode D indexes

| | Full Barcode | Nucleic acid |
|---|---|---|
| | sequence | index sequence |
| 6mer | 5′-TTTTTTTTTTTTTTT | TGTGGT |
| | TGTGGT | (SEQ ID NO: 48) |
| | (SEQ ID NO: 13) | |
| 7mer | 5′-TTTTTTTTTTTTTTT | TGTGGTG |
| | TGTGGTG | (SEQ ID NO: 49) |
| | (SEQ ID NO: 14) | |
| 8mer | 5′-TTTTTTTTTTTTTTT | TGTGGTGT |
| | TGTGGTGT | (SEQ ID NO: 50) |
| | (SEQ ID NO: 15) | |
| 9mer | 5′-TTTTTTTTTTTTTTT | TGTGGTGTT |
| | TGTGGTGTT | (SEQ ID NO: 51) |
| | (SEQ ID NO: 16) | |
| Probe | 5′-AF647-AAC ACC ACA | |
| | (SEQ ID NO: 32) | |

TABLE 5
Barcode E indexes

| | Full Barcode | Nucleic acid |
|---|---|---|
| | sequence | index sequence |
| 6mer | 5′-TTTTTTTTTTTTTTT | GCTTCT |
| | GCTTCT | (SEQ ID NO: 52) |
| | (SEQ ID NO: 17) | |
| 7mer | 5′-TTTTTTTTTTTTTTT | GCTTCTT |
| | GCTTCTT | (SEQ ID NO: 53) |
| | (SEQ ID NO: 18) | |
| 8mer | 5′-TTTTTTTTTTTTTTT | GCTTCTTC |
| | GCTTCTTC | (SEQ ID NO: 54) |
| | (SEQ ID NO: 19) | |
| 9mer | 5′-TTTTTTTTTTTTTTT | GCTTCTTCC |
| | GCTTCTTCC | (SEQ ID NO: 55) |
| | (SEQ ID NO: 20) | |
| Probe | 5′-AF488-TT GGA AGA AGC | |
| | (SEQ ID NO: 33) | |

TABLE 6
Barcode F indexes

| | Full Barcode | Nucleic acid |
|---|---|---|
| | sequence | index sequence |
| 6mer | 5′-TTTTTTTTTTTTTTT | GGTTCT |
| | GGTTCT | (SEQ ID NO: 56) |
| | (SEQ ID | |
| | NO: 21) | |
| 7mer | 5′-TTTTTTTTTTTTTTT | GGTTCTC |
| | GGTTCTC | (SEQ ID NO: 57) |
| | (SEQ ID | |
| | NO: 22) | |
| 8mer | 5′-TTTTTTTTTTT | GGTTCTCT |
| | GGTTCTCT | (SEQ ID NO: 58) |
| | (SEQ ID | |
| | NO: 23) | |
| 9mer | 5′-TTTTTTTTTTTTTTT | GGTTCTCTG |
| | GGTTCTCTG | (SEQ ID NO: 59) |
| | (SEQ ID NO: 24) | |
| Probe | 5′-AF594-CAG AGA ACC | |
| | (SEQ ID NO: 34) | |

The residence time for the timers was very short (˜500 ms), the times for the 7mer (˜1 s) and 8mer (˜10 s) were longer, and the 9mer had the longest residence time (˜25 s). Table 7 shows all measured residence times. For Barcode A, the 7mer exhibited a 3.75-fold increase in residence time relative to the timer, the 8mer exhibited a 3-fold increase in residence time relative to the 7mer, and the 9mer exhibited a 1.9-fold increase in residence time relative to the 8mer. For Barcode B, the 7mer exhibited a 3.6-fold increase in residence time relative to the 6mer, the 8mer exhibited a 3.3-fold increase in residence time relative to the 7mer, and the 9mer exhibited a 3.8-fold increase in residence time relative to the 8mer. For Barcode C, the 7mer exhibited a 7-fold increase in residence time relative to the 6mer, the 8mer exhibited a 5.6-fold increase in residence time relative to the 7mer, and the 9mer exhibited a 4.9-fold increase in residence time relative to the 8mer. For Barcode D, the 8mer exhibited a 4.3-fold increase in residence time relative to the 7mer, and the 9mer exhibited a 3.2-fold increase in residence time relative to the 8mer. For Barcode E, the 7mer exhibited a 9-fold increase in residence time relative to the 6mer, the 8mer exhibited a 2-fold increase in residence time relative to the 7mer, and the 9mer exhibited a 1.7-fold increase in residence time relative to the 8mer.

For Barcode F, the 9mer exhibited a 16-fold increase in residence time relative to the 7mer.

TABLE 7
Barcode residence times
On Times (seconds)

| Barcode | 6mer | 7mer | 8mer | 9mer |
|---|---|---|---|---|
| A | 0.8 ± 0.4 | 3.0 ± 1.5 | 9.0 ± 2.7 | 17.0 ± 3.2 |
| B | 1.1 ± 0.3 | 4.0 ± 1.3 | 13.0 ± 3.1 | 50.0 ± 10.0 |
| C | 0.2 ± 0.1 | 1.4 ± 0.3 | 7.7 ± 2.2 | 38.0 ± 5.9 |
| D | N/A | 1.2 ± 0.1 | 5.2 ± 0.2 | 16.6 ± 1.0 |
| E | 0.3 ± 0.1 | 2.7 ± 0.2 | 5.8 ± 0.3 | 9.8 ± 1.3 |
| F | N/A | 1.2 ± 0.0 | N/A | 19.7 ± 4.5 |

### Example 2. Molecular Barcode Assembly and Readout

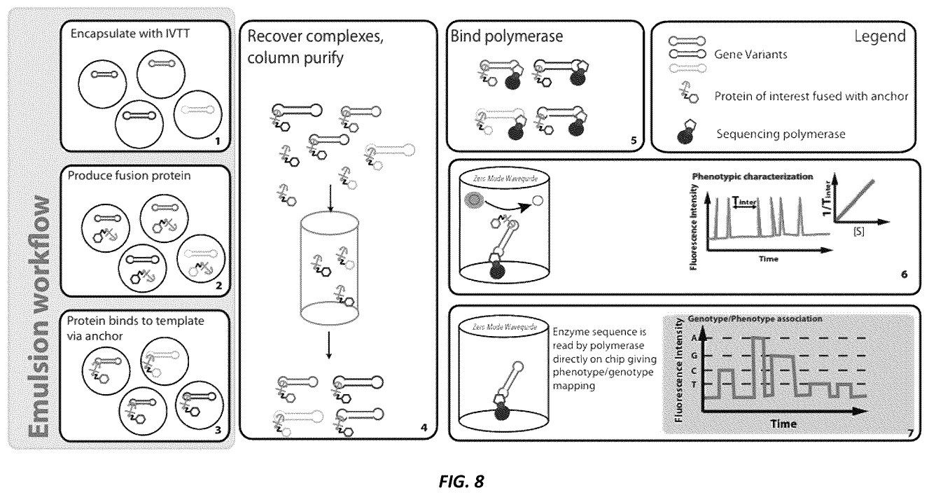

This example relates to the creation, by ligation, of a double-stranded DNA molecular barcode comprising multiple DNA indexes. Each index was a stretch of ˜10 (order of magnitude) nucleotides that represents one sequence of a number of possible sequences. The barcodes were captured on a solid glass surface in a microfluidic flow cell. It was demonstrated that the indexes can be exposed by nicking and subsequent dehybridization of the “lid” (FIG. 6). In addition, a single-stranded binding protein was added to the molecular barcode to improve efficiency by maintaining the rigidity of the double-stranded structure, avoiding the collapse to a random coil structure that would hide the indexes (FIG. 6). The barcode indexes were then identified by observing, at the single-molecule level, the transient hybridization of fluorescently-labeled nucleic acid- or protein-based probes. Through tagging with fluorophores, each probe was associated with one color (or a combination of colors) or one fluorescence lifetime (FIG. 7). Moreover, each probe was associated with multiple indexes and result in different binding kinetics, Table 8.

TABLE 8
Example index sequences.

| | |
|---|---|
| Probe Sequence | GAGCAGCAG |
| | (SEQ ID NO: 35) |
| Index 1 Sequence | CTCGTCGTC |
| | (slow kinetics) |
| | (SEQ ID NO: 25) |
| Index 2 Sequence | CTCGTCGTA |
| | (medium kinetics) |
| | (SEQ ID NO: 26) |
| Index 3 Sequence | CTCGTCGAA |
| | (fast kinetics) |
| | (SEQ ID NO: 27) |
| Index 4 Sequence | CTCGTCAAA |
| | (ultrafast kinetics) |
| | (SEQ ID NO: 28) |

The timescale of the transient hybridization depended on the length of the index. A Ent index had a probe binding lifetime of ˜100 ms, whereas a 9 nt barcode had a binding lifetime of ˜30 s. These different kinetics were observable and allowed for the identification of the associated index. Therefore, six colors (blue (B), red (R; ACAAGGAGA), green (G), B+R, B+G, R+G B+R+G) gave a total of 7×4=28 different possible combinations with one index, or ˜200 combinations with 2 different indexes which are read in parallel and possibly thousands with three indexes. Indexes were combined to decode 2-index barcodes using two-color imaging (type B1+R1), or by creating type R1+R3 which give traces with two levels.

### Example 3. Screening for One or More Characteristics of a Protein

This example describes the use of droplet microfluidics/single-molecule approach for the directed evolution of proteins (e.g., proteins having an enzymatic activity) (FIG. 8). A pool of DNA molecules encoding for a fusion protein comprising a protein variant fused to a DNA binding domain such as an inactivated meganuclease is provided. As shown in FIG. 8(1), individual DNA molecules are encapsulated in individual microcompartments (fL volume aqueous droplets) using a microfluidic device together with an enzyme mix for in vitro transcription translation (IVTT). The microcompartment size is such that only one of ten compartments contains a DNA molecule. Specifically, using pM nucleic acid concentrations provides that most droplets contain either a single nucleic acid molecule or no molecule at all. The microcompartments are then incubated off-chip (separate from a microfluidic device) to allow for transcription and translation to produce the fusion proteins (FIG. 8(2)). Following translation, the DNA binding domain of the translated protein spontaneously binds to an anchor on the DNA molecule and compartmentalization ensures that each protein variant binds to the DNA molecule coding for that specific variant (FIG. 8(3)). The emulsion is then broken in the presence of heparin, which quickly binds and sequesters the excess (unbound) enzymes from the IVTT mix, and complexes of protein bound to the DNA molecule coding for said protein are recovered by size exclusion chromatography (FIG. 8(4)). A sequencing polymerase is incubated with the recovered complexes to allow for a direct readout of the protein, and the DNA molecule provides a binding site to which the sequencing polymerase can be bound (FIG. 8(5)). The complexes are then loaded on a zero-mode waveguide (ZMW) sequencing chip. A phenotypic assay (e.g., an enzymatic assay) is performed to identify one or more characteristics of each protein variant (FIG. 8(6)). Enzymatic kinetics on one (or multiple) substrates of the protein variants are observed by single-molecule fluorescence. In a second stage the polymerase is activated by the addition of dNTPs and the sequence of the protein is read directly on the sequencing chip to provide a complete phenotype/genotype mapping.

### Example 4. Single-Molecule Selection of Antibodies

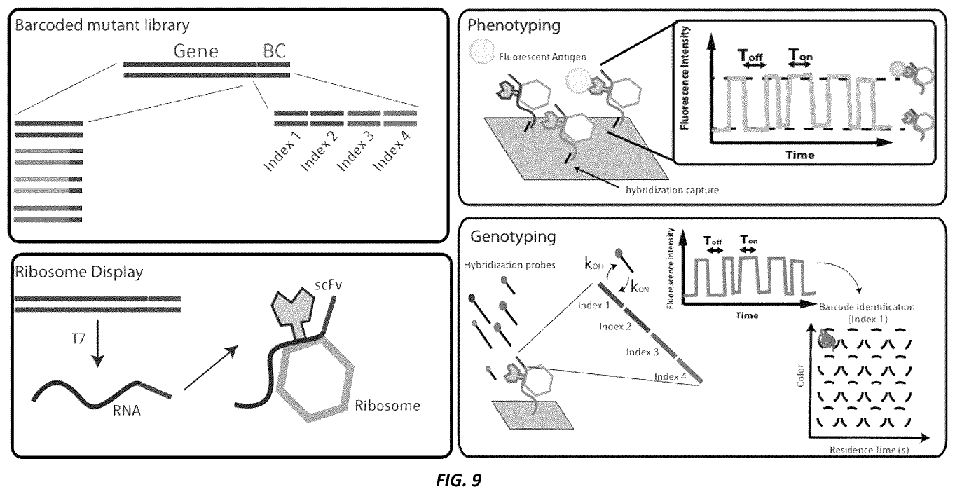

This example describes a single-molecule approach for the evolution of single chain variant fragments (scFv) of antibodies (FIG. 9). A pool of DNA molecules, each encoding for scFv variants, is provided. Each gene is labeled with a short barcode composed of four indexes chosen from a small alphabet of highly dissimilar sequences. A set of scFv/ribosome/RNA complexes are expressed by ribosome display. The complexes are immobilized on a glass slide and the binding/unbinding dynamics (Kₒₙ and K_{off} for each scFv) of a fluorescent antigen is observed for each complex. A set of hybridization probes for each barcode index is introduced in the microfluidic chamber. One of the probes, the one complementary to the specific sequence in the first index, exhibits binding/unbinding dynamics indicative of the identity of the index. The probe and thus the sequence of the index is identified by color and residence time (proportional to probe length). Four different barcode indexes, with four different colored fluorophores and four probe lengths (hence four residence times) for each index, allows for ˜10⁵ combinations to be decoded.

### Example 5. Single-Molecule Selection of RNA Aptamers

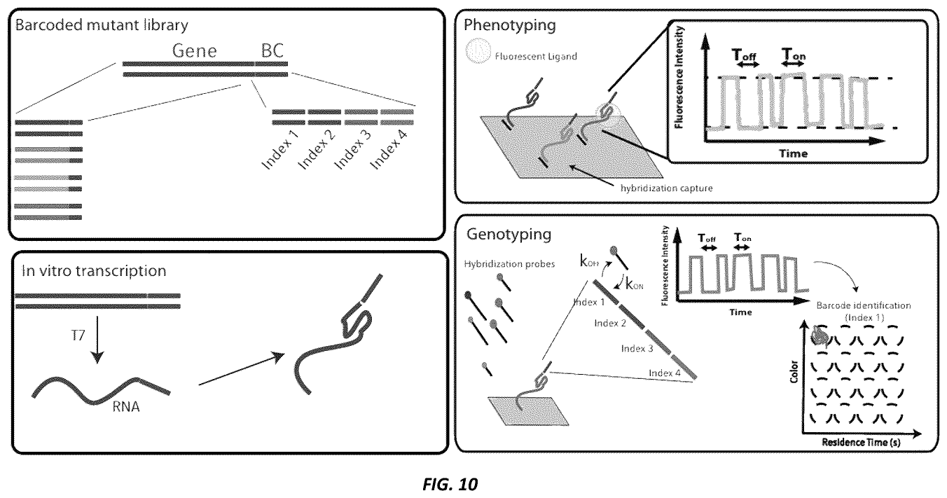

This example describes a single-molecule approach for the evolution of RNA aptamers (FIG. 10). First, a pool of DNA molecules encoding for aptamer variants was provided. Each gene was labeled with a short barcode composed of four indexes chosen from a small alphabet of highly dissimilar sequences. RNA aptamers were produced by in vitro transcription. The aptamers were bound to a glass slide and, for each one, the binding/unbinding dynamics (Kₒₙ and K_{off} for each aptamer) of a fluorescent ligand was measured. For each barcode index, a set of hybridization probes was introduced in the microfluidic chamber. One of the probes, the one complementary to the specific sequence in the index, exhibited binding/unbinding dynamics. The probe and thus the sequence of index one was identified by color and residence time (proportional to probe length). Four different barcode indexes, with four different colored fluorophores and four probe lengths (hence four residence times) for each index allows for ˜10⁵ combinations to be decoded.

### Example 6. Polymerase Screening by Single-Molecule Fluorescence

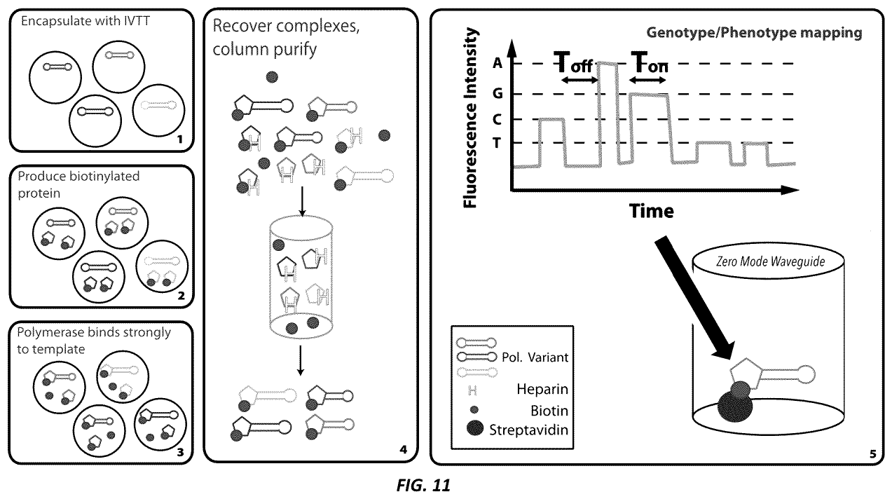

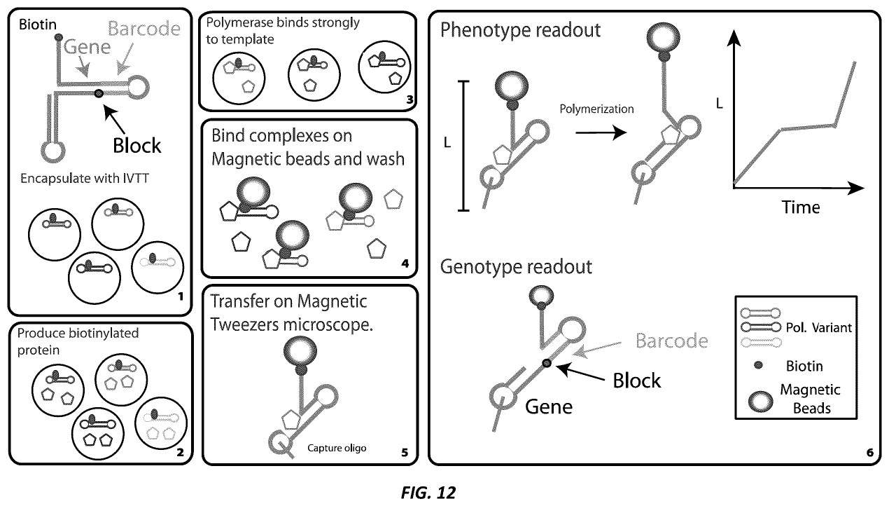

This example describes a single-molecule approach based on droplet microfluidics for the evolution of polymerases (FIG. 11). First, a pool of dumbbell shaped DNA molecules that encode for polymerase variants is provided. Using a microfluidic device, each molecule is encapsulated in a microcompartment (a fL volume aqueous droplet) together with an enzyme mix for both in vitro transcription and translation (IVTT) and biotinylation. Microcompartment size and DNA concentration are such that only one out of ten compartments contain a DNA molecule. Using pM nucleic acid concentrations provides that most droplets contain either a single nucleic acid molecule or no molecule at all. This ensures the absence of multiple DNA molecule per compartment, and thus phenotype to genotype mapping. The microcompartments are incubated off-chip (separate from the microfluidic device) to allow for transcription, translation and biotinylation of each DNA molecule. The polymerase spontaneously binds to the dumbbells to form a stable complex but is unable to replicate DNA due to the absence of one or more dNTPs. Compartmentalization ensures that each DNA molecule is bound by its respective encoded polymerase. The emulsion is then broken in the presence of heparin which quickly binds and sequesters the excess, i.e., unbound, polymerase. DNA-polymerase complexes are then recovered by size exclusion chromatography. Complexes can be loaded on a sequencing chip and the behavior of single DNA polymerases can be observed at high resolution. The phenotype of the enzyme is given by the kinetics of the sequencing traces while the genotype is directly read out from the sequencing data (either by directly sequencing the polymerase gene, or sequencing an associated DNA barcode).

Enzymatic next-generation sequencing (NGS) applications require a polymerase that a) is able to incorporate modified nucleotides, b) has long processivity, i.e., can replicate long stretches of DNA before detaching from the template strand, c) has kinetic parameters well adapted to the imaging technology and d) is resistant to photodamage. While polymerases can be easily engineered to accept modified nucleotides by standard methods, the other properties are difficult to engineer in bulk. Rational design coupled to trial-and-error is currently the most successful strategy but is slow and costly.

In order to select enzymes possessing all these features at the same time, enzyme/DNA complexes are formed in microcompartments (fL volume aqueous droplets). To do so, DNA molecules coding for the enzyme of interest are provided under the form of DNA dumbbells. Using a microfluidic design, the pool of DNA molecules are encapsulated in microcompartments, together with a cell-free expression system and a biotinylation enzyme and substrates (FIG. 11). Due to the small size of the microcompartments, 10⁷ different DNA molecules can be put into complexes using less than 15 μl of material in total, reducing costs with respect to bulk experiments. The microcompartments are incubated at constant temperature leading to the transcription and translation of the DNA molecules and to the biotinylation of the translated enzyme (FIG. 11). The polymerase spontaneously form a strong complex with the DNA molecule by binding to the single stranded loop region, maintaining a genotype/phenotype link (FIG. 11). The DNA/enzyme complexes are purified using size exclusion chromatography (FIG. 11) before being loaded on ZMW. The sequencing activity is then measured for each complex separately and is directly dependent of the variant polymerase sequence. The method is innovative in part due to the fact that the sequencing data reveals concomitantly the sequence of each polymerase (either by directly sequencing the polymerase gene or sequencing an associated DNA barcode) and its kinetic properties, allowing the selection of the best-suited enzymes.

### Example 7. Polymerase Screening by Magnetic Tweezers

This example describes a single-molecule approach based on droplet microfluidics for the screening of polymerases via magnetic tweezers (FIG. 12). First, a pool of DNA (or RNA) containing the gene of the polymerase of interest and a sequence associated barcode is provided. Each gene contains a hairpin structure with one branch ending in a biotin and the other in a loop. The gene and the barcode are separated by a C12 connection, a roadblock for polymerization. Using a microfluidic device, each molecule is encapsulated in a microcompartment (a fL volume aqueous droplet) together with an enzyme mix for both in vitro transcription and translation (IVTT). Microcompartment size and DNA concentration are such that only one out of ten compartments contain a DNA molecule. This ensures the absence of multiple DNA molecule per compartment. The microcompartments are incubated off-chip to allow for transcription and translation. The polymerase spontaneously binds to the hairpins to form a stable complex but is unable to replicate DNA due to the absence of dNTPs. Compartmentalization ensures that each molecule is bound by its respective encoded polymerase. The emulsion and the complexes are bound on streptavidin covered beads. Complexes were loaded on a magnetic tweezers microscope so that the behavior of single DNA polymerases could be observed at high resolution. The phenotype of the enzyme is given by the kinetics of the trace while the genotype is directly read by mechanically sequencing the associated barcode.

### Example 8. Polymerase Directed Evolution

This example describes the identification of polymerases with desired phenotypes by directed evolution. A method to perform parallel in vitro transcription and translation (IVTT) and biotinylation was developed (FIG. 11). This method was validated with a DNA polymerase. The method works in bulk, i.e., when the DNA molecules are not individually compartmentalized, as well as in emulsion.

The recovery of active polymerase DNA from a bulk IVTT reaction has been demonstrated. In this experiment, DNA molecules coding for different polymerases (Q1, active and QDead, inactive) were re-suspended in an IVTT/biotinylation mixture and encapsulated in microcompartments on a microfluidic chip. The DNA concentration was such that only one in ten droplets contains a DNA molecule. The DNA was transcribed and translated to protein with compartments keeping genotype/phenotype association. These polymerases have strong affinity for DNA (Kd≈50 pM) and form complexes with their own genes. The gene itself has a high molecular weight (1300 kDa) so that enzyme/DNA complexes can easily be separated from the rest of the proteins of the IVTT/biotinylation mixture as well as from the excess polymerases not bound to any template. The recovery of intact complexes can be tested by resuspending the complexes in a buffer suitable for DNA replication and subsequently quantifying the amplification by quantitative PCR. As expected in absence of crosstalk, the results show how only the complexes of Q1, the active polymerase, are amplified. This suggests that stable complexes can be formed in emulsions and recovered after emulsion breaking.

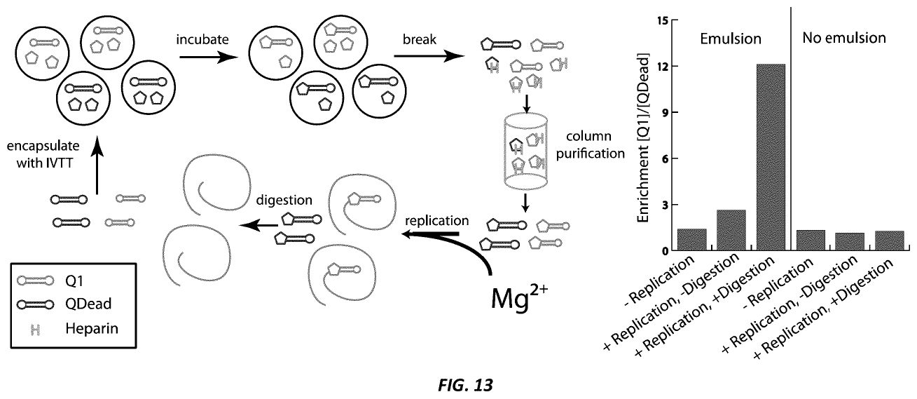

A mix of DNA molecules encoding either for active (Q1) or inactive mutant (Qdead) polymerase was encapsulated in microcompartments, with one molecule per 10 microcompartments on average. The compartments also contained an IVTT enzyme mix so that DNA molecules were transcribed and translated in emulsion droplets. Translated polymerase enzymes were observed to bind to their own template. The emulsion was then broken in the presence of heparin that quickly binds to the excess (unbound) polymerase and sequesters it. Intact complexes were purified by size exclusion and re-suspended in a buffer containing dNTPs but lacking Mg²⁺. The addition of Mg²⁺ triggered replication of the active complexes while the inactive complexes stayed unreplicated. The input DNA (DpnI Methylated) can be further removed by DpnI digestion. The extent of the differential amplification of Q1 over QDead can be quantified by qPCR. FIG. 13 shows the enrichment, i.e., the concentration of Q1 genes over QDead genes. Replication plus digestion led to a 12-fold increase of Q1 over QDead. This effect is not found in bulk experiments, thus demonstrating the role of compartmentalization.

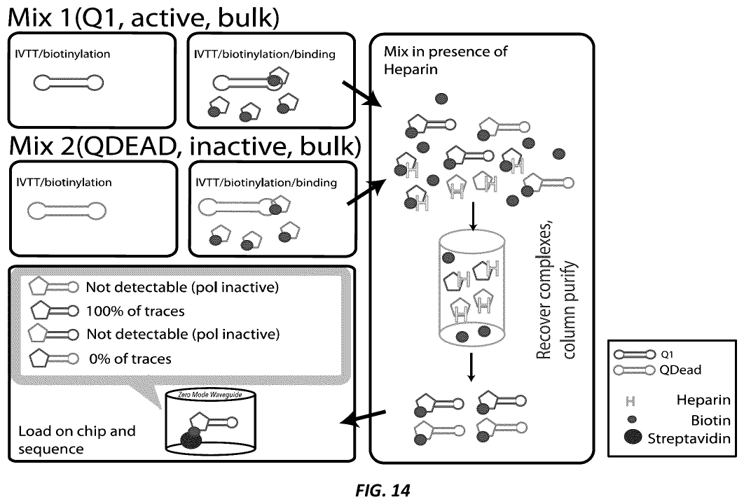

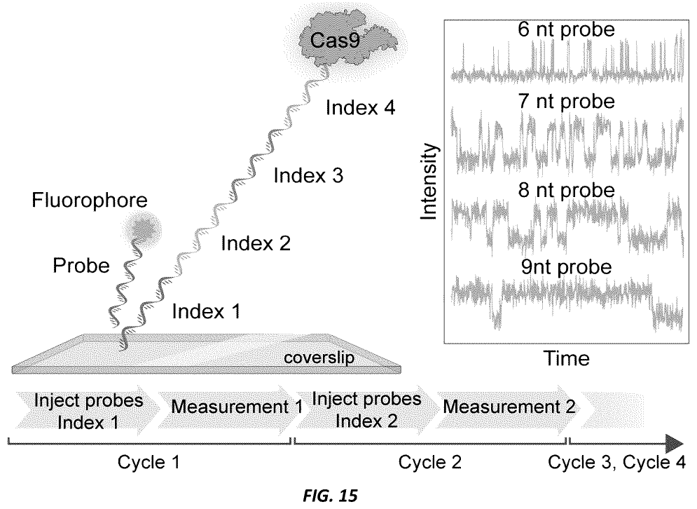

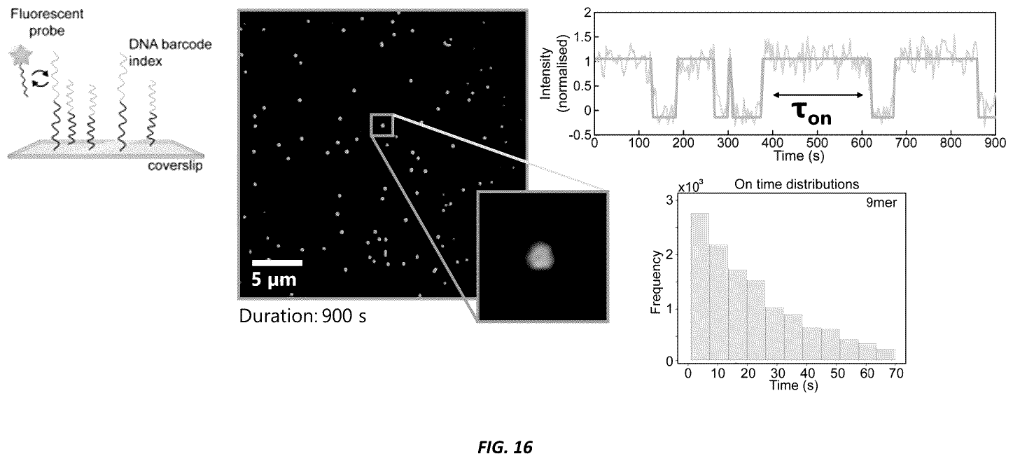

In a further experiment, it was demonstrated that complexes formed in the IVTT/biotinylation mix can be directly loaded on ZMW and used in sequencing experiments. In this experiment, two separate bulk reactions were prepared with DNA molecules coding for either an active polymerase (Q1) or an inactivated version (QDead, binds DNA but cannot replicate) in the IVTT/biotinylation enzymes (FIG. 14). After transcription, translation and binding of the enzyme to the DNA template, the two reactions were mixed in the presence of heparin, which quickly binds to excess polymerases so as to avoid the binding of polymerase to DNA other than their own gene. The polymerase/DNA complexes were then purified by size exclusion and loaded on a sequencing chip (PacBio). This allowed for testing of two important aspects of the workflow: 1) enzyme/DNA complexes produced in IVTT are suitable for direct loading on chip and 2) there is no crosstalk between different templates in the mixing of the two reactions, a step that mimics emulsion breaking (FIG. 11). In fact, the two polymerases used here have radically different phenotypes (active/inactive). Crosstalk in the reaction would amount to finding active polymerases sequencing the gene corresponding to the inactive polymerases. This does not happen in the experiment showing that phenotype/genotype association can be maintained across the whole workflow.

Finally, two different bulk reactions were prepared with DNA molecules coding for either an active (Q1, green) or an inactive (QDead, red) together with an IVTT/biotinylation enzyme cocktail. After transcription, translation and biotinylation, the protein bound to the DNA molecule formed a stable enzyme/DNA complex. The two reactions were mixed in the presence of heparin (that sequesters excess, unbound polymerases) and the complexes were purified by size exclusion. Purified complexes can then be loaded directly on a chip for analysis by single-molecule sequencing.

### Example 9. Characterization of Index Binding Kinetics

This example relates to the design of molecular barcodes and characterization of index binding kinetics. Similar to Example 1, six orthogonal index sequences were generated. These nucleic acid index sequences are adapted to specifically bind a 9-nucleotide (nt) fluorescently-labeled probe. The longest index for each sequence has a 9-nt complementarity with the probe. Additional index lengths for each sequence (7 and 8 nt) were also generated. A probe can bind to all four index lengths; however, the residence times (binding kinetics) of the probe depends on the length of the sequence overlap, allowing for discrimination and identification of the nucleic acid index sequences relative to one another.

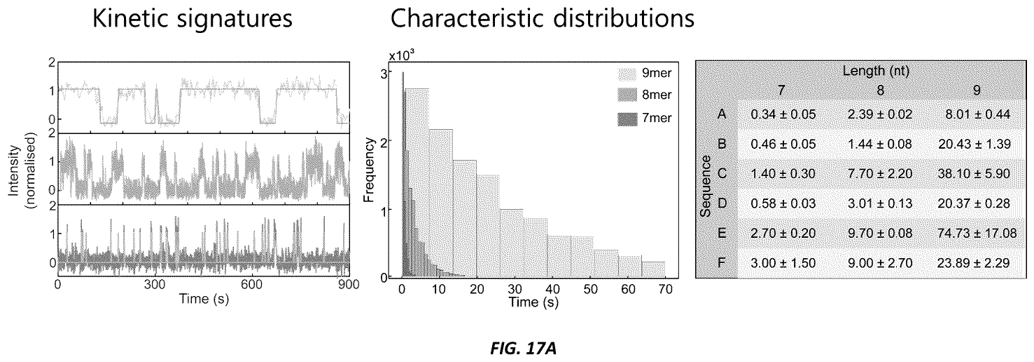

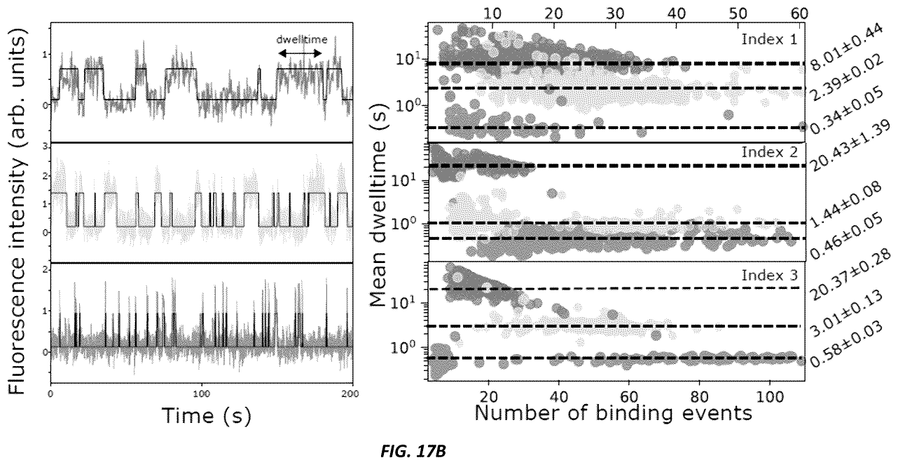

As shown in FIG. 17A, the residence times for the 7 nt length sequences were the shortest for each of Sequence A, B, C, D, E, and F, while the residence times for the 9 nt length sequences were the longest. These data demonstrate that changes in the lengths of index sequences can allow for differentiation of discrete molecular barcode sequences when using the same nucleic acid probe (e.g., fluorescently labeled probe).

### Example 10. Identification of a Target Analyte

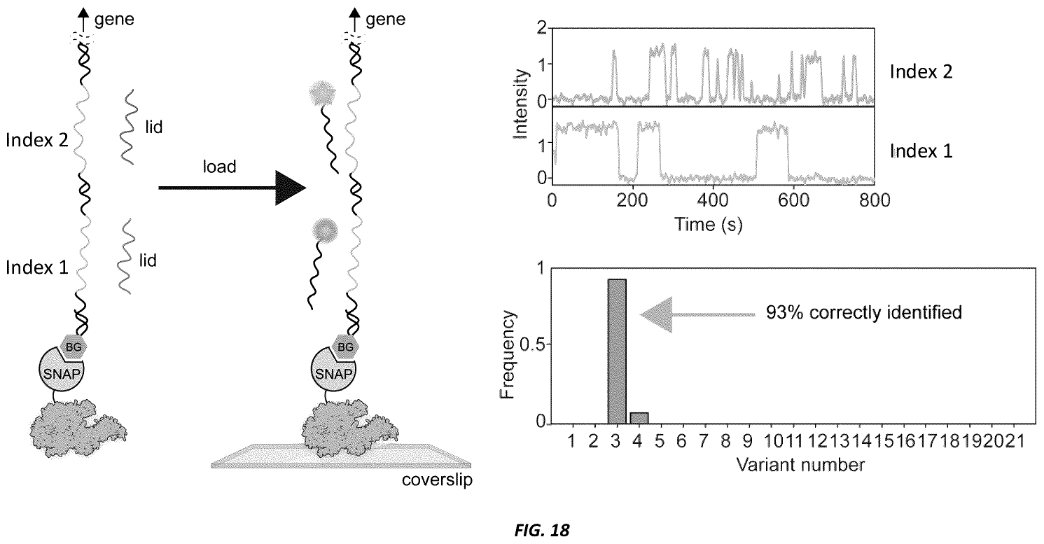

A target analyte was covalently attached to a double-stranded nucleic acid barcode (dsDNA barcode) comprising a first nucleic acid index sequence (Index 1) and a second nucleic acid index sequence (Index 2) separated by a nucleic acid spacer. The barcode was attached to a coverslip via a secondary interaction (BG-SNAP). The barcode was exposed to a nicking enzyme that generated nicks in one strand of the barcode at restriction sites surrounding the index sequences (e.g., on each side of the index sequences). In the presence of SSB protein, the segments of the nucleic acid bound to the index sequences (“lid” sequences) and nicked by the nicking enzyme were removed/dehybridized to generate a rigid nucleic acid barcode comprising exposed index sequences. Two fluorescently labeled probes were added to the target analyte, wherein one of the probes was complementary to Index 1 and the other was complementary to Index 2. Residence times were measured by observing the fluorescence over a period of at least 800 seconds. The measurements correctly identified the target analyte as being Variant #3 in 93% of experimental determinations. See FIG. 18. These data demonstrate that even as few as two nucleic acid probes and two index sequences can be used to correctly identify a target analyte.

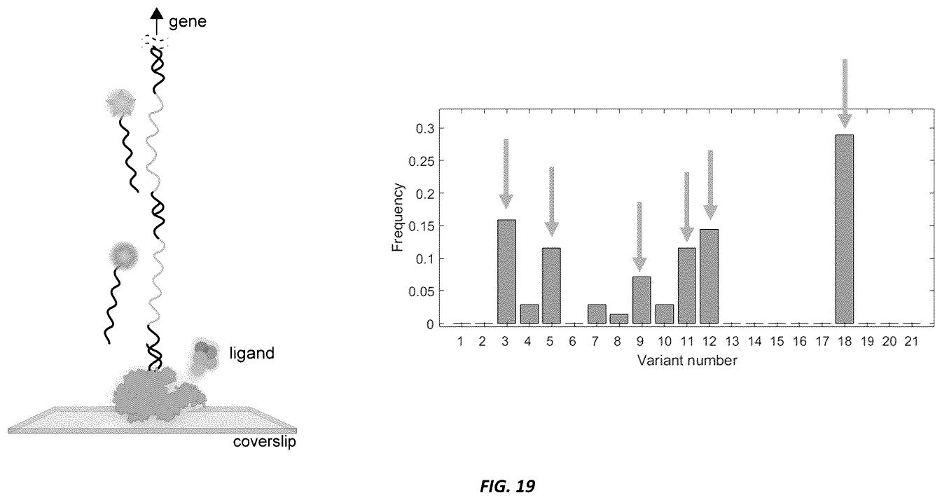

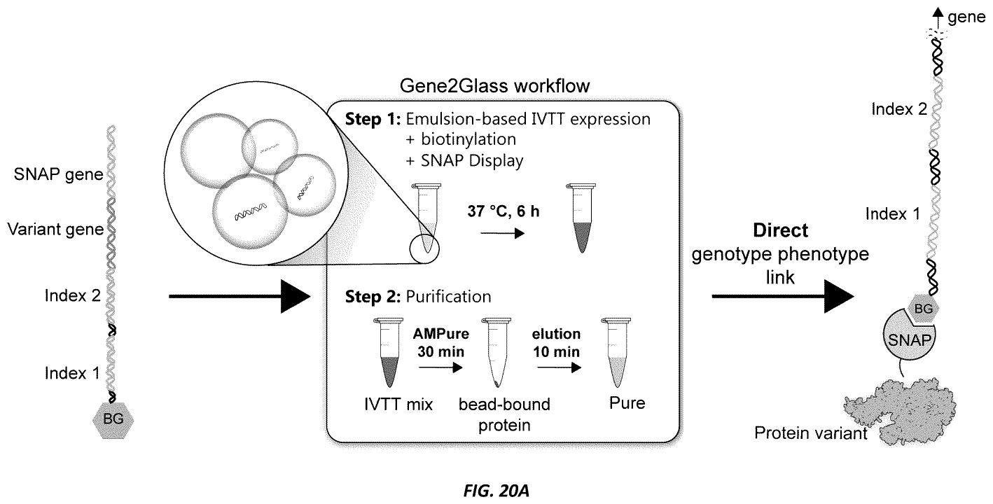

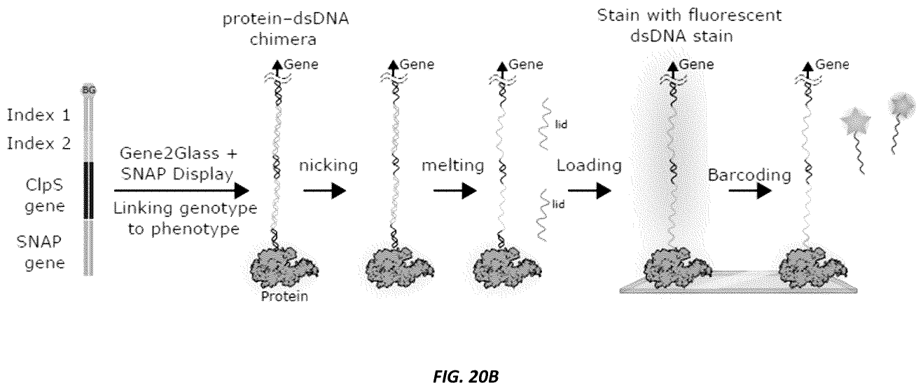

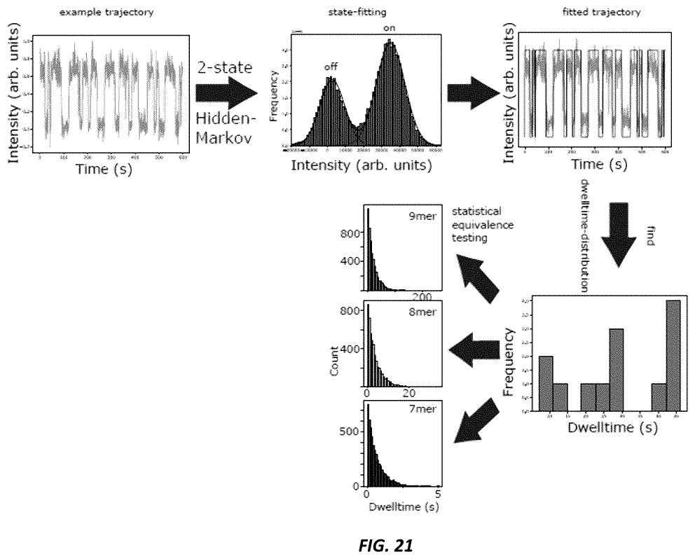

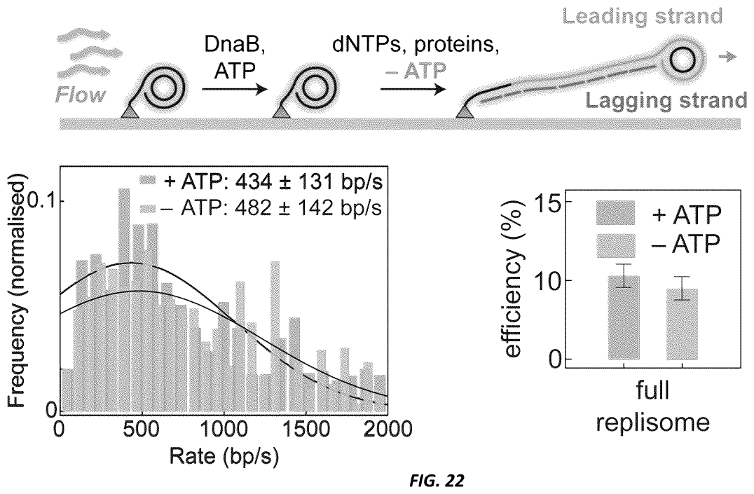

A similar experiment was performed to identify multiple protein analytes within a single experiment (FIG. 19). Each analyte was attached to a different nucleic acid barcode. The same two fluorescently labeled probes were able to correctly identify at least six discrete analytes.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A method comprising:
contacting an analyte with a first barcode recognition molecule and a second barcode recognition molecule, wherein the analyte is connected to a barcode comprising a first nucleic acid index sequence and a second nucleic acid index sequence, wherein the first barcode recognition molecule specifically binds to the first nucleic acid index sequence and the second barcode recognition molecule specifically binds to the second nucleic acid index sequence;
detecting a series of signal pulses indicative of binding interactions between (i) the first barcode recognition molecule and the first nucleic acid index sequence, and (ii) the second barcode recognition molecule and the second nucleic acid index sequence; and
determining the identity of the analyte based on the series of signal pulses.

2. A method comprising:
contacting an analyte with a first barcode recognition molecule and a second barcode recognition molecule, wherein the analyte is connected to a barcode comprising a first nucleic acid index sequence, wherein the first barcode recognition molecule and the second barcode recognition molecule specifically bind to the first nucleic acid index sequence, wherein either (a) the first barcode recognition molecule binds to the first nucleic acid index sequence with a different affinity than the second barcode recognition molecule binds to the first nucleic acid index sequence or (b) the first barcode recognition molecule comprises a first detectable label and the second barcode recognition molecule comprises a second detectable label;
detecting a series of signal pulses indicative of binding interactions between (i) the first barcode recognition molecule and the first nucleic acid index sequence, and (ii) the second barcode recognition molecule and the first nucleic acid index sequence; and determining the identity of the analyte based on the series of signal pulses.

3. A method comprising:
(i) contacting an analyte with a first barcode recognition molecule and a second barcode recognition molecule, wherein the analyte is connected to a double-stranded barcode comprising a first nucleic acid index sequence and a second nucleic acid index sequence, wherein the first barcode recognition molecule specifically binds to the first nucleic acid index sequence and the second barcode recognition molecule specifically binds to the second nucleic acid index sequence;
(ii) detecting a series of signal pulses indicative of binding interactions between (i) the first barcode recognition molecule and the first nucleic acid index sequence, and (ii) the second barcode recognition molecule and the second nucleic acid index sequence; and
(iii) determining the identity of the analyte based on the series of signal pulses.

4. A method comprising:
(i) contacting an analyte with a first barcode recognition molecule, wherein the analyte is connected to a double-stranded barcode comprising a first nucleic acid index sequence and a second nucleic acid index sequence, wherein the first barcode recognition molecule specifically binds to the first nucleic acid index sequence and specifically binds to the second nucleic acid index sequence;
(ii) detecting a series of signal pulses indicative of binding interactions between (i) the first barcode recognition molecule and the first nucleic acid index sequence, and (ii) the first barcode recognition molecule and the second nucleic acid index sequence; and
(iii) determining the identity of the analyte based on the series of signal pulses.

5. The method of claim 3 or 4, wherein, prior to (i), one or more segments of the nucleic acid strand that is bound to the index sequences are removed from the double-stranded barcode, optionally wherein this contacting step is performed in the presence of single-stranded binding (SSB) protein.

6. The method of claim 5, wherein the one or more segments of the nucleic acid strand that is bound to the index sequences are removed from the double-stranded barcode using incubation with enzymes or chemical means.

7. The method of claim 3 or 4, wherein, prior to (i), the double-stranded barcode is contacted with a nicking enzyme to remove one or more segments of the nucleic acid strands that is bound to the index sequences, optionally wherein the double-stranded barcode comprises restriction sites surrounding the index sequences that are recognized by the nicking enzyme, optionally wherein this contacting step is performed in the presence of single-stranded binding (SSB) protein.

8. The method of any one of claims 5-7, wherein the SSB protein is a herpes simplex virus (HSV-1) single-strand DNA-binding protein, a bacterial SSB, replication protein A, or Eukaryotic mitochondrial SSB.

9. The method of any one of the preceding claims, wherein the analyte is a DNA molecule, an RNA molecule, a polypeptide, a protein, or a nucleic acid aptamer.

10. The method of any one of the preceding claims, wherein the first nucleic acid index sequence and the second nucleic acid index sequence comprise different nucleotide sequences and/or different nucleic acid modifications.

11. The method of any one of the preceding claims, wherein the first nucleic acid index sequence and/or the second nucleic acid index sequence comprises a length of 4-15 nucleotides, 5-10 nucleotides, 6-9 nucleotides, 6 nucleotides, 7 nucleotides, 8 nucleotides, or 9 nucleotides.

12. The method of any one of the preceding claims, wherein the nucleotide sequence of the second nucleic acid index sequence comprises one nucleobase substitution relative to the nucleotide sequence of the first nucleic acid index sequence.

13. The method of any one of the preceding claims, wherein the barcode comprises a spacer between the first nucleic acid index sequence and the second nucleic acid index sequence.

14. The method of claim 13, wherein the spacer is a non-nucleic acid spacer or a nucleic acid spacer, optionally wherein the non-nucleic acid spacer is a polyethylene glycol spacer.

15. The method of claim 14, wherein the nucleic acid spacer comprises a length of 5-35 nucleotides, 10-25 nucleotides, 15-30 nucleotides, 10-20 nucleotides, 10, 11, 12, 13, 14, 15, 16, 17, 18, 19, 20, or 25 nucleotides.

16. The method of any one of the preceding claims, wherein the barcode further comprises a third nucleic acid index sequence, optionally wherein the third nucleic acid index sequence comprises two nucleobase substitutions relative to the nucleotide sequence of the first nucleic acid index sequence.

17. The method of claim 16, wherein the first barcode recognition molecule specifically binds to the third nucleic acid index sequence.

18. The method of claim 16, wherein the analyte is contacted with a third barcode recognition molecule, wherein the third barcode recognition molecule specifically binds to the third nucleic acid index sequence.

19. The method of any one of claims 16-18, wherein the barcode further comprises a fourth nucleic acid index sequence, optionally wherein the fourth nucleic acid index sequence comprises three nucleobase substitutions relative to the nucleotide sequence of the first nucleic acid index sequence.

20. The method of claim 19, wherein the first barcode recognition molecule specifically binds to the fourth nucleic acid index sequence.

21. The method of claim 19, wherein the analyte is contacted with a fourth barcode recognition molecule, wherein the fourth barcode recognition molecule specifically binds to the fourth nucleic acid index sequence.

22. The method of any one of the preceding claims, wherein the signal pulse comprises a pulse duration that is characteristic of a dissociation rate of binding between the barcode recognition molecule and a nucleic acid index sequence.

23. The method of any one of the preceding claims, wherein at least one signal pulse is separated from another by an interpulse duration that is characteristic of an association rate of barcode recognition molecule binding.

24. The method of any one of the preceding claims, wherein the barcode recognition molecule is an oligonucleotide probe, a nucleic acid aptamer, or a protein.

25. The method of claim 24, wherein the oligonucleotide probe comprises a length of 4-15 nucleotides, 5-10 nucleotides, 6-9 nucleotides, 6 nucleotides, 7 nucleotides, 8 nucleotides, or 9 nucleotides.

26. The method of any one of the preceding claims, wherein at least one of the barcode recognition molecules comprises a detectable label.

27. The method of claim 26, wherein the detectable label is a luminescent label, a fluorescent label, or a conductivity label.

28. The method of claim 27, wherein the luminescent label is a fluorophore or a dye.

29. The method of any one of claims 26-28, wherein the first barcode recognition molecule, the second barcode recognition molecule, the third barcode recognition molecule and/or the fourth barcode recognition molecule comprise different detectable labels.

30. The method of any one of claims 26-29, wherein the first barcode recognition molecule and the second barcode recognition molecule comprise different detectable labels.

31. The method of claim 30, wherein the detectable labels of the first barcode recognition molecule and the second barcode recognition molecule are fluorophores, and wherein the fluorophore of the first barcode recognition molecule and the fluorophore of the second barcode recognition molecule comprise different absorption/emission spectral properties, optionally wherein the fluorophore of the first barcode recognition molecule and the fluorophore of the second barcode recognition molecule produce different colors.

32. The method of any one of claims 26-31, wherein at least one of the barcode recognition molecules further comprises a quencher molecule.

33. The method of claim 32, wherein the quencher molecule quenches the signal from the detectable label when the barcode recognition molecule is not bound to a nucleic acid index sequence, but does not quench the signal from the detectable label when the barcode recognition molecule is bound to a nucleic acid index sequence.

34. The method of any one of the preceding claims, wherein the series of signal pulses is a series of real-time signal pulses.

35. The method of any one of the preceding claims, wherein the analyte is attached to a surface, optionally a glass or silica-based surface.

36. The method of claim 35, wherein the surface is a surface of a well of a multi-well plate, optionally a 96-well plate or a 384-well plate.

37. The method of claim 35 or 36, wherein the analyte is covalently or non-covalently attached to the surface.

38. The method of any one of claims 35-37, wherein the analyte is attached to the surface via a secondary molecule or species, optionally via a streptavidin-biotin linkage or by hybridization to a capture oligonucleotide probe covalently linked to the surface.

39. The method of any one of the preceding claims, wherein the analyte is contacted with the first barcode recognition molecule and the second barcode recognition molecule simultaneously.

40. The method of any one of preceding claims, wherein the analyte is contacted with all of the barcode recognition molecules simultaneously.

41. The method of any one of claims 1-37, wherein:
(a) the analyte is first contacted with the first barcode recognition molecule and the series of signal pulses indicative of binding interactions between the first barcode recognition molecule and the nucleic acid index sequence(s) are detected; and
(b) the analyte is subsequently contacted with the second barcode recognition molecule and the series of signal pulses indicative of binding interactions between the second barcode recognition molecule and the nucleic acid index sequence(s) are detected.

42. A method comprising:
(i) attaching a biomolecule to a surface, wherein the biomolecule comprises (a) a protein and a barcode comprising a first nucleic acid index sequence, or (b) an aptamer and a barcode comprising a first nucleic acid index sequence;
(ii) performing a phenotypic assay to determine one or more characteristics of the protein or aptamer;
(iii) contacting the biomolecule with a first barcode recognition molecule, wherein the first barcode recognition molecule specifically binds to the first nucleic acid index sequence;
(iv) detecting a series of signal pulses indicative of binding interactions between the first barcode recognition molecule and the first nucleic acid index sequence;
(v) determining the identity of the biomolecule based on the series of signal pulses.

43. A method comprising:
(i) attaching a biomolecule to a surface, wherein the biomolecule comprises (a) a nucleic acid comprising a coding sequence encoding a protein and a molecular barcode comprising a first nucleic acid index sequence, and (b) an amino acid sequence of the protein;
(ii) performing a phenotypic assay to determine one or more characteristics of the protein;
(iii) contacting the biomolecule with a first barcode recognition molecule, wherein the first barcode recognition molecule specifically binds to the first nucleic acid index sequence;
(iv) detecting a series of signal pulses indicative of binding interactions between the first barcode recognition molecule and the first nucleic acid index sequence;
(v) determining the identity of the biomolecule based on the series of signal pulses.

44. The method of claim 42 or 43, wherein the barcode further comprises a second nucleic acid index sequence.

45. The method of claim 44, wherein the first barcode recognition molecule specifically binds to the second nucleic acid index sequence.

46. The method of claim 44, wherein the biomolecule is contacted with a second barcode recognition molecule in (iii), wherein the second barcode recognition molecule specifically binds to the second nucleic acid index sequence.

47. The method of any one of claims 44-46, wherein the first nucleic acid index sequence and the second nucleic acid index sequence comprise different nucleotide sequences and/or different nucleic acid modifications.

48. The method of any one of claims 42-47, wherein the first nucleic acid index sequence and/or the second nucleic acid index sequence comprises a length of 4-15 nucleotides, 5-10 nucleotides, 6-9 nucleotides, 6 nucleotides, 7 nucleotides, 8 nucleotides, or 9 nucleotides.

49. The method of any one of claims 44-48, wherein the nucleotide sequence of the second nucleic acid index sequence comprises one nucleobase substitution relative to the nucleotide sequence of the first nucleic acid index sequence.

50. The method of any one of claims 44-49, wherein the barcode comprises a spacer between the first nucleic acid index sequence and the second nucleic acid index sequence.

51. The method of claim 50, wherein the spacer is a non-nucleic acid spacer or a nucleic acid spacer, optionally wherein the non-nucleic acid spacer is a polyethylene glycol spacer.

52. The method of claim 51, wherein the nucleic acid spacer comprises a length of 5-35 nucleotides, 10-25 nucleotides, 15-30 nucleotides, 10-20 nucleotides, 10, 11, 12, 13, 14, 15, 16, 17, 18, 19, 20, or 25 nucleotides.

53. The method of any one of claims 44-52, wherein the barcode further comprises a third nucleic acid index sequence, optionally wherein the third nucleic acid index sequence comprises two nucleobase substitutions relative to the nucleotide sequence of the first nucleic acid index sequence.

54. The method of claim 53, wherein the first barcode recognition molecule specifically binds to the third nucleic acid index sequence.

55. The method of claim 53, wherein the biomolecule is contacted with a third barcode recognition molecule in (iii), wherein the third barcode recognition molecule specifically binds to the third nucleic acid index sequence.

56. The method of any one of claims 53-55, wherein the barcode further comprises a fourth nucleic acid index sequence, optionally wherein the fourth nucleic acid index sequence comprises three nucleobase substitutions relative to the nucleotide sequence of the first nucleic acid index sequence.

57. The method of claim 56, wherein the first barcode recognition molecule specifically binds to the fourth nucleic acid index sequence.

58. The method of claim 56, wherein the biomolecule is contacted with a fourth barcode recognition molecule in (iii), wherein the fourth barcode recognition molecule specifically binds to the fourth nucleic acid index sequence.

59. The method of any one of claims 42-58, wherein the signal pulse comprises a pulse duration that is characteristic of a dissociation rate of binding between the barcode recognition molecule and a nucleic acid index sequence.

60. The method of any one of claims 42-59, wherein at least one signal pulse is separated from another by an interpulse duration that is characteristic of an association rate of barcode recognition molecule binding.

61. The method of any one of claims 42-60, wherein the barcode recognition molecule is an oligonucleotide probe, a nucleic acid aptamer, or a protein.

62. The method of claim 61, wherein the oligonucleotide probe comprises a length of 4-15 nucleotides, 5-10 nucleotides, 6-9 nucleotides, 6 nucleotides, 7 nucleotides, 8 nucleotides, or 9 nucleotides.

63. The method of any one of claims 42-62, wherein at least one of the barcode recognition molecules comprises a detectable label.

64. The method of claim 63, wherein the detectable label is a luminescent label, a fluorescent label, or a conductivity label.

65. The method of claim 64, wherein the luminescent label is a fluorophore or a dye.

66. The method of any one of claims 63-65, wherein the first barcode recognition molecule, the second barcode recognition molecule, the third barcode recognition molecule and/or the fourth barcode recognition molecule comprise different detectable labels.

67. The method of any one of claims 63-66, wherein the first barcode recognition molecule and the second barcode recognition molecule comprise different detectable labels.

68. The method of claim 67, wherein the detectable labels of the first barcode recognition molecule and the second barcode recognition molecule are fluorophores, and wherein the fluorophore of the first barcode recognition molecule and the fluorophore of the second barcode recognition molecule comprise different absorption/emission spectral properties, optionally wherein the fluorophore of the first barcode recognition molecule and the fluorophore of the second barcode recognition molecule produce different colors.

69. The method of any one of claims 63-68, wherein at least one of the barcode recognition molecules further comprises a quencher molecule.

70. The method of claim 69, wherein the quencher molecule quenches the signal from the detectable label when the barcode recognition molecule is not bound to a nucleic acid index sequence, but does not quench the signal from the detectable label when the barcode recognition molecule is bound to a nucleic acid index sequence.

71. The method of any one of claims 42-70, wherein the series of signal pulses is a series of real-time signal pulses.

72. The method of any one of claims 42-71, wherein the surface is a glass or silica-based surface.

73. The method of claim 72, wherein the surface is a surface of a well of a multi-well plate, optionally a 96-well plate or a 384-well plate.

74. The method of claim 71 or 72, wherein the biomolecule is covalently or non-covalently attached to the surface.

75. The method of any one of claims 42-74, wherein the biomolecule is attached to the surface via a secondary molecule or species, optionally via a streptavidin-biotin linkage or by hybridization to a capture oligonucleotide probe covalently linked to the surface.

76. The method of any one of claims 42-75, wherein the biomolecule further comprises a spacer between the protein and the barcode.

77. The method of any one of claims 42-75, wherein the biomolecule further comprises a spacer between the aptamer and the barcode.

78. The method of any one of claims 43-75, wherein the biomolecule further comprises a ribosome attached to the coding sequence.

79. The method of any one of claim 43-75 or 78, wherein the coding sequence is attached to the amino acid sequence.

80. The method of any one of claims 42-79, wherein the phenotypic assay is a binding assay or an enzymatic assay.

81. The method of claim 80, wherein the binding assay comprises incubating the biomolecule with an antigen or ligand, optionally a fluorescently labeled antigen or ligand, and determining the binding affinity of the biomolecule for the antigen or ligand.

82. The method of claim 80, wherein the enzymatic assay comprises incubating the biomolecule with substrate, and determining the ability of the biomolecule to chemically convert said substrate and/or determining the enzymatic activity of the biomolecule.

83. The method of any one of claims 42-79, wherein the one or more characteristics of the protein or aptamer comprise binding affinity for an antigen, binding kinetics, stability of the protein or aptamer, thermal stability of the protein or aptamer, and/or enzymatic activity number.

84. A method of screening protein variants and/or aptamer variants comprising:
(i) generating a library of biomolecules, wherein each of the biomolecules comprises (a) a protein variant and a molecular barcode comprising a first nucleic acid index sequence, or (b) an aptamer variant and a molecular barcode comprising a first nucleic acid index sequence, wherein each of the biomolecules comprises a unique combination of first nucleic acid index sequences;
(ii) attaching each of the biomolecules to a surface;
(iii) performing a phenotypic assay to determine one or more characteristics of each of the protein variants;
(iv) contacting each of the biomolecules with a first barcode recognition molecule, wherein the first barcode recognition molecule specifically binds to the first nucleic acid index sequence;
(v) detecting a series of signal pulses indicative of binding interactions between the first barcode recognition molecule and the first nucleic acid index sequence;
(vi) determining the identity of each of the biomolecules based on the series of signal pulses; and
(vii) identifying biomolecules comprising unique protein variants having one or more desired characteristics.

85. The method of any one of claims 42-84, wherein the biomolecule is contacted with the first barcode recognition molecule and the second barcode recognition molecule simultaneously.

86. The method of any one of claims 42-85, wherein the biomolecule is contacted with all of the barcode recognition molecules simultaneously.

87. The method of any one of claims 42-84, wherein:
(a) the biomolecule is first contacted with the first barcode recognition molecule and the series of signal pulses indicative of binding interactions between the first barcode recognition molecule and the nucleic acid index sequence(s) are detected; and
(b) the biomolecule is subsequently contacted with the second barcode recognition molecule and the series of signal pulses indicative of binding interactions between the second barcode recognition molecule and the nucleic acid index sequence(s) are detected.

88. The method of claim 84, wherein the library comprises 5-1000, 5-500, 5-100, 10-100, 100-1000, or 50-500 unique biomolecules.

89. A nucleic acid barcode comprising two, three, or four nucleic acid index sequences, wherein each of the nucleic acid index sequences is independently selected from any one of SEQ ID NOs: 25-28 or 36-59.

90. A nucleic acid barcode comprising a first nucleic acid index sequence, a second nucleic acid index sequence, a third nucleic acid index sequence, and a fourth nucleic acid index sequence, wherein each of the nucleic acid index sequences comprises at least 6 nucleotides in length, and wherein the second nucleic acid index sequence comprises one nucleobase substitution relative to the first nucleic acid index sequence, the third nucleic acid index sequence comprises two nucleobase substitutions relative to the first nucleic acid index sequence, and the fourth nucleic acid index sequence comprises three nucleobase substitutions relative to the first nucleic acid index sequence.

91. A nucleic acid barcode comprising a first nucleic acid index sequence, a second nucleic acid index sequence, a third nucleic acid index sequence, and a fourth nucleic acid index sequence, wherein each of the nucleic acid index sequences is complementary to a barcode recognition molecule,
wherein the residence time for a binding interaction between the second nucleic acid index sequence and the barcode recognition molecule is at least 2-fold greater than the binding interaction between the first nucleic acid index sequence and the barcode recognition molecule, wherein the residence time for a binding interaction between the third nucleic acid index sequence and the barcode recognition molecule is at least 2-fold greater than the binding interaction between the second nucleic acid index sequence and the barcode recognition molecule, and
wherein the residence time for a binding interaction between the fourth nucleic acid index sequence and the barcode recognition molecule is at least 2-fold greater than the binding interaction between the fourth nucleic acid index sequence and the barcode recognition molecule.

92. The nucleic acid barcode of claim 91, wherein the residence time for a binding interaction between the second nucleic acid index sequence and the barcode recognition molecule is 2-fold to 5-fold greater than the binding interaction between the first nucleic acid index sequence and the barcode recognition molecule,
wherein the residence time for a binding interaction between the third nucleic acid index sequence and the barcode recognition molecule is 2-fold to 5-fold greater than the binding interaction between the second nucleic acid index sequence and the barcode recognition molecule, and
wherein the residence time for a binding interaction between the fourth nucleic acid index sequence and the barcode recognition molecule is 2-fold to 5-fold greater than the binding interaction between the fourth nucleic acid index sequence and the barcode recognition molecule.

93. The nucleic acid barcode of claim 91 or 92, wherein the residence time for a binding interaction between the second nucleic acid index sequence and the barcode recognition molecule is 2-fold, 3-fold, 4-fold, or 5-fold greater than the binding interaction between the first nucleic acid index sequence and the barcode recognition molecule,
wherein the residence time for a binding interaction between the third nucleic acid index sequence and the barcode recognition molecule is 2-fold, 3-fold, 4-fold, or 5-fold greater than the binding interaction between the second nucleic acid index sequence and the barcode recognition molecule, and
wherein the residence time for a binding interaction between the fourth nucleic acid index sequence and the barcode recognition molecule is 2-fold, 3-fold, 4-fold, or 5-fold greater than the binding interaction between the fourth nucleic acid index sequence and the barcode recognition molecule.

94. The nucleic acid barcode of any one of claims 89-93, wherein nucleic acid index sequences comprise a length of 4-15 nucleotides, 5-10 nucleotides, 6-9 nucleotides, 6 nucleotides, 7 nucleotides, 8 nucleotides, or 9 nucleotides.

95. The nucleic acid barcode of any one of claims 89-94, wherein the barcode comprises a spacer between the first nucleic acid index sequence and the second nucleic acid index sequence.

96. The nucleic acid barcode of claim 95, wherein the spacer is a non-nucleic acid spacer, optionally a polyethylene glycol spacer, or a nucleic acid spacer.

97. The nucleic acid barcode of claim 96, wherein the nucleic acid spacer comprises a length of 5-25 nucleotides, 10-20 nucleotides, 10, 11, 12, 13, 14, 15, 16, 17, 18, 19, or 20 nucleotides.


SEQUENCE LISTING (59 sequences)

SEQ ID NO: 1 - DNA, 21 bp, synthetic construct
  source 1..21: mol_type = other DNA
ttttttttttttttttgttcc

SEQ ID NO: 2 - DNA, 22 bp, synthetic construct
  source 1..22: mol_type = other DNA
ttttttttttttttttgttcct

SEQ ID NO: 3 - DNA, 23 bp, synthetic construct
  source 1..23: mol_type = other DNA
ttttttttttttttttgttcctc

SEQ ID NO: 4 - DNA, 24 bp, synthetic construct
  source 1..24: mol_type = other DNA
ttttttttttttttttgttcctct

SEQ ID NO: 5 - DNA, 21 bp, synthetic construct
  source 1..21: mol_type = other DNA
tttttttttttttttctctcc

SEQ ID NO: 6 - DNA, 22 bp, synthetic construct
  source 1..22: mol_type = other DNA
tttttttttttttttctctccc

SEQ ID NO: 7 - DNA, 23 bp, synthetic construct
  source 1..23: mol_type = other DNA
tttttttttttttttctctccct

SEQ ID NO: 8 - DNA, 24 bp, synthetic construct
  source 1..24: mol_type = other DNA
tttttttttttttttctctccctc

SEQ ID NO: 9 - DNA, 21 bp, synthetic construct
  source 1..21: mol_type = other DNA
tttttttttttttttgcttgt

SEQ ID NO: 10 - DNA, 22 bp, synthetic construct
  source 1..22: mol_type = other DNA
tttttttttttttttgcttgtc

SEQ ID NO: 11 - DNA, 23 bp, synthetic construct
  source 1..23: mol_type = other DNA
tttttttttttttttgcttgtct

SEQ ID NO: 12 - DNA, 24 bp, synthetic construct
  source 1..24: mol_type = other DNA
tttttttttttttttgcttgtctc

SEQ ID NO: 13 - DNA, 21 bp, synthetic construct
  source 1..21: mol_type = other DNA
ttttttttttttttttgtggt

SEQ ID NO: 14 - DNA, 22 bp, synthetic construct
  source 1..22: mol_type = other DNA
ttttttttttttttttgtggtg

SEQ ID NO: 15 - DNA, 23 bp, synthetic construct
  source 1..23: mol_type = other DNA
ttttttttttttttttgtggtgt

SEQ ID NO: 16 - DNA, 24 bp, synthetic construct
  source 1..24: mol_type = other DNA
ttttttttttttttttgtggtgtt

SEQ ID NO: 17 - DNA, 21 bp, synthetic construct
  source 1..21: mol_type = other DNA
tttttttttttttttgcttct

SEQ ID NO: 18 - DNA, 22 bp, synthetic construct
  source 1..22: mol_type = other DNA
tttttttttttttttgcttctt

SEQ ID NO: 19 - DNA, 23 bp, synthetic construct
  source 1..23: mol_type = other DNA
tttttttttttttttgcttcttc

SEQ ID NO: 20 - DNA, 24 bp, synthetic construct
  source 1..24: mol_type = other DNA
tttttttttttttttgcttcttcc

SEQ ID NO: 21 - DNA, 21 bp, synthetic construct
  source 1..21: mol_type = other DNA
tttttttttttttttggttct

SEQ ID NO: 22 - DNA, 22 bp, synthetic construct
  source 1..22: mol_type = other DNA
tttttttttttttttggttctc

SEQ ID NO: 23 - DNA, 23 bp, synthetic construct
  source 1..23: mol_type = other DNA
tttttttttttttttggttctct

SEQ ID NO: 24 - DNA, 24 bp, synthetic construct
  source 1..24: mol_type = other DNA
tttttttttttttttggttctctg

SEQ ID NO: 25-29 - skipped (5 entries)

SEQ ID NO: 30 - DNA, 11 bp, synthetic construct
  source 1..11: mol_type = other DNA
ttgagggagag

SEQ ID NO: 31 - DNA, 11 bp, synthetic construct
  source 1..11: mol_type = other DNA
ttgagacaagc

SEQ ID NO: 32 - skipped

SEQ ID NO: 33 - DNA, 11 bp, synthetic construct
  source 1..11: mol_type = other DNA
ttggaagaagc

SEQ ID NO: 34-59 - skipped (26 entries)